(19) United States
(12) Patent Application Publication
(10) Pub. No.: US 2007/0098693 A1
(43) Pub. Date: May 3, 2007

(54) Methods for enhancing engraftment of purified hematopoietic stem cells in allogenic recipients

(76) Inventor: Suzanne T. Ildstad, Prospect, KY (US)

Correspondence Address: FISH & RICHARDSON P.C., PO BOX 1022, MINNEAPOLIS, MN, 55440-1022, US

(21) Appl. No.: 10/558,516

(22) PCT Filed: May 28, 2004
(86) PCT No.: PCT/US04/16843

§ 371 Date: Dec. 5, 2006

Related U.S. Application Data
(60) Provisional application No. 60/473,829, filed on May 28, 2003.

(51) Int. Cl. A61K 35/14; A61K 31/66; A61N 5/00

(52) U.S. Cl. 424/93.7; 514/109; 600/1

(57) ABSTRACT

CD8⁺/TCR⁻ bone marrow cells facilitate engraftment of hemapoietic stem cells (HSQ in allogeneic recipients without causing graft versus host disease. The present invention identifies the main subpopulation (55-65%) of CD8⁺/TCR⁻ facilitating cells (FQ as plasmacytoid precursor dendritic cells (p-preDC). The present invention notably demonstrates that FC and p-preDC share many phenotypic, morphological, functional features, including IFN-α production, activation and survival after stimulation, and expansion and maturation after FIO-Ligand (FL) treatment. FL mobilized FC, the majority of which express a pre-DC phenotype, facilitate HSC engraftment. Although p-preDC significantly enhance HSC engraftment, they do so with less efficiency than FC. The present invention for the first time defines a direct functional role for p-preDC in HSC engraftment and will have a significant impact on strategies to design effective facilitating cell-based therapies for transplantation.

### CROSS-REFERENCE TO RELATED APPLICATIONS

This application claims priority to U.S. Provisional Application Ser. No. 60/473,829, filed May 28, 2003, which is incorporated herein by reference in its entirety.

### CONTRACTUAL ORIGIN OF THE INVENTION

This invention was supported in part by NIH grant no. 5 R01 HL063443-03 and ______, awarded by the National Institutes of Health. The government has certain rights to this invention.

### BACKGROUND OF THE INVENTION

1. Field of the Invention

The present invention is directed toward novel cell-based therapeutic strategies to optimize the composition of a graft in order to reduce the morbidity of HSC transplants in mismatched recipients. More specifically, the present invention relates to compositions comprising FL-induced FC and their use in reducing morbidity of HSC transplants.

2. Description of the Prior Art

Throughout this application, various references are referred to within parentheses. Disclosures of these publications in their entireties are hereby incorporated by reference into this application to more fully describe the state of the art to which this invention pertains. Full bibliographic citation for these references may be found at the end of this application, preceding the claims.

An elusive goal in organ transplantation and the treatment of autoimmune diseases is the induction of tolerance. Bone marrow chimerism induces robust donor-specific tolerance to organ and cellular transplants and, as such, offers significant therapeutic potential^{1,2}. However, graft-versus-host disease (GVHD) and the toxicity of ablative conditioning have limited the widespread clinical application of this approach³⁻⁴. Mature donor T cells are the primary cells responsible for GVHD. T cell depletion (TCD) of bone marrow prevents GVHD, but is associated with significantly impaired engraftment⁵⁻⁷. The inventor of the present invention has identified, and others have confirmed, a CD8⁺TCR⁻ population that facilitates hematopoietic stem cell (HSC) engraftment across major histocompatibility complex (MHC) barriers without inducing GVHD⁸⁻¹⁰. These bone marrow CD8⁺/TCR⁻ facilitating cells (FC) are comprised of a heterogenous population and share cell surface markers with T cells, but are distinct from T cells¹¹. FC also share phenotypic characteristics with CD8α lymphoid dendritic cells (DCs)⁸

The role of DCs in transplantation has recently become the focus of intense research, with the emerging concept of using “tolerogenic” DC in the graft to silence host immunity and enhance engraftment, while also preventing GVHD^{12, 13}. DC have the unique capacity to activate or tolerize naïve T cells¹⁴⁻¹⁶. Immature DCs capture and process foreign antigens (Ag) in peripheral tissues, up-regulate co-stimulatory molecules, and migrate to lymphoid organs. Mature DCs present the processed Ag to naïve and resting T cells and induce an antigen-specific immune response. Besides their immunogenic function, DCs play a key role in the induction of immunological tolerance by tolerizing donor T cells to self antigen^{17,18}. The broad functions of DC (immunization vs tolerization) can be explained by: 1) the recent identification of distinct DC subsets; 2) the dose, nature and duration of the activation signals received by the DC and, 3) the maturation state of DC upon encounter with antigen^{15, 19}. It has been proposed that the presence of interleukin-10 (IL-10) during the maturation of DC results in a shift in DC phenotype and that the IL-10-modulated DC, or “tolerogenic” DC, mediate tolerance by inducing anergic and regulatory T cells in transplantation. Moreover, direct evidence for the tolerogenic role of DC came from studies showing that the administration of DC (either immature DC or mature CD8α⁺ lymphoid DC) before fully MHC-mismatched cardiac allograft transplantation prolonged graft survival^{14,26-29}.

Recently, the role of plasmacytoid DC precursors (p-preDC) in transplantation has become a topic of major interest. Recent findings led to the proposal that p-preDC are “tolerogenic” DC since the presence of these cells in the bone marrow graft correlated with a decreased occurrence of GVHD²²⁻²⁴. Initially, p-preDC were described as the major type 1-interferon (IFN)-producing cells (IPCs) due to their potent capacity to produce IFN-α in response to virus or to microbial stimulation with TLR9 ligands such as CpG ODN (an immunostimulatory bacterial DNA sequence rich in CpG) in vitro²⁵⁻²⁷. Plasmacytoid pre-DC represent the most important effector cell of the anti-viral innate immune system, and is the precursor for the antigen-presenting cell critical for initiating adaptive immune responses^{28, 29}. However, p-preDC can induce the development of either a Th1 or a Th2 immune response, depending on the dose and/or the nature of antigen exposure¹⁹. Murine p-preDC are a rare, bone marrow-derived B220⁺/CD11c^{dim}/CD11b⁻ cell population with a plasmacytoid morphology. In HSC transplantation, a direct functional role for p-preDC has not yet been defined.

Hematopoietic stem cell (HSC) chimerism has the potential to treat autoimmune disease, hemoglobinopathies, and to induce tolerance to organ and islet allografts. However, the widespread application of this approach is limited by graft-versus-host disease (GVHD). Attempts have therefore been made to identify cells with facilitative potential with the goal to enhance engraftment with reduced toxicity.

A number of groups have characterized bone marrow cell populations that facilitate HSC engraftment in allogeneic recipients [9, 51-57]. Kaufman et al. first described CD8⁺/TCR⁻ facilitating cells (FC) that enhanced engraftment without causing GVHD [9]. As few as 10,000 FC facilitated engraftment of purified HSC in ablated allogeneic recipients conditioned with 950 cGy total body irradiation (TBI) [9]. In this model, CD8⁺ T cells could not substitute for FC function. Transplantation of CD8⁺/TCR⁻ FC alone did not radioprotect, eliminating the possibility that FC themselves had HSC properties [9]. Notably, when recipients were conditioned with a less ablative dose (800 cGy) of TBI, both CD8⁺/TCR⁺ and CD8⁺/TCR⁻ cells were required to facilitate HSC engraftment [8].

Schuchert et al. demonstrated that CD8⁺/TCR⁻ FC express a CD3-receptor complex containing the TCR-β chain disulfide linked to a 33 kDa protein that is neither TCR-α nor pre-Tα [48]. When FC were obtained from RAG2^{-/-} and TCR-β^{-/-} donors, they exhibited impaired function, further supporting a role for the CD3ε associated 33 kDa FC complex [48]. Until now, the ontogeny of FC has not been defined. In fact, the presence of T cell markers on FC has led to some question whether the biological activity of FC is due to contaminating T lymphocytes. If conventional T cells are critical to facilitation, the GVHD-producing capacity of these cells would severely limit the clinical application of HSC chimerism as a therapeutic modality.

The present invention utilizes molecular and genetic analyses to determine if the requirements for the development of functional CD8⁺/TCR⁻ FC are different from the requirements for T cell development. The present invention shows for the first time that although HSC-derived, FC are distinct functionally and developmentally from T cells. FC contain transcripts for CD3ε and CD3δ, but not TCRα or TCRβ. FC obtained from CD3ε mutant donors are not functional, suggesting that the CD3 complex may have a critical role in FC action in allogeneic transplantation. FC also enhance engraftment of HSC in syngeneic recipients. Importantly, bone marrow CD8⁺ T cells fail to facilitate in syngeneic engraftment, further delineating functional differences between FC and T cells. The inclusion of FC in grafts may provide an attractive approach to enhance potency, and reduce toxicity, especially when the number of HSC required for engraftment is suboptimal.

### SUMMARY OF THE INVENTION

The present invention identifies a definitive role for p-preDC in facilitating function in the CD8⁺/TCR⁻FC population. The present invention further demonstrates that the majority of FC share phenotypic characteristics with p-preDC and exhibit a similar plasmacytoid morphology. Notably, FC resemble p-preDC functionally in their ability to secrete IFN-α, and other pro-inflammatory cytokines, mature by up-regulating activation markers exhibit increased survival after activation by CpG ODN. Recombinant human Flt-3 Ligand (FL), a key cytokine for p-preDC development^{27, 30 31}, similarly regulates FC in that FC can be generated from FL-supplemented BM cell cultures, as well as expanded and mobilized in vivo in FL-treated mice. More than 90% of FL-mobilized FC express CD11c⁺ (a dendritic cell marker) and a large majority exhibit a p-preDC phenotype. Additionally, these mobilized FC facilitated long-term HSC engraftment and induced tolerance in allogeneic recipient mice.

Because of the similarities between p-preDC and FC, the present inventor examined whether p-preDC contribute directly to HSC facilitation in vivo. The present invention shows for the first time that p-preDC do significantly facilitate HSC engraftment. However, the p-preDC facilitate HSC engraftment less efficiently than FC total, suggesting that FC consist of p-preDC that act in concert with other collaborative cell types to allow optimal HSC engraftment. A clear definition of FC phenotype and mechanism of action may allow for a promising cell-based approach to enhance engraftment and tolerance while avoiding alloreactivity.

The present invention further demonstrates for the first time that FC development and function is independent of T cells and cannot be replaced by them. Purified GFP⁺ HSC transplanted in syngeneic recipients produce GFP⁺ FC which facilitate in secondary transplants, confirming that FC are derived from HSC. Moreover, FC develop prior to T cells after HSC transplantation, again indicating that they are separate from T cells. In addition, FC, but not T cells, potently facilitate the engraftment of suboptimal numbers of HSC in syngeneic recipients. Notably, FC contain the transcripts for CD3ε and CD3δ, but not TCRα or TCRβ, indicating a non-T-cell lineage derivation and excluding the possibility of T cell contamination. Genetic mutations that generate a functional deficiency in CD3 signaling significantly impair FC function in allogeneic facilitation (P=0.006).

The present invention further demonstrates for the first time that FC development and function is independent of T cells and cannot be replaced by them. Purified GFP⁺ HSC transplanted in syngeneic recipients produce GFP⁺ FC which facilitate in secondary transplants, confirming that FC are derived from HSC. Moreover, FC develop prior to T cells after HSC transplantation, again indicating that they are separate from T cells. In addition, FC, but not T cells, potently facilitate the engraftment of suboptimal numbers of HSC in syngeneic recipients. Notably, FC contain the transcripts for CD3ε and CD3δ, but not TCRα or TCRβ, indicating a non-T-cell lineage derivation and excluding the possibility of T cell contamination. Genetic mutations that generate a functional deficiency in CD3 signaling significantly impair FC function in allogeneic facilitation (P=0.006).

### BRIEF DESCRIPTION OF THE FIGURES

The accompanying drawings, which are incorporated herein and form a part of the specification, illustrate non-limiting embodiments of the present invention, and together with the description, serve to explain the principles of the invention.

In the Figures:

FIG. 1a: CD8⁺/TCR⁻ FC: a heterogeneous population: CD11c⁺ FC are the predominant subpopulation in sorted FC. BM cells stained with anti-αβ-TCR FITC, anti-γδ-TCR FITC and anti-CD8α-PE were isolated from the lymphoid gate (intermediate forward scatter and lower side scatter, R1) and sorted for CD8α⁺/TCRαβ⁻/TCRγδ⁻ (FC gate). The sorted FC were blocked using the anti-Fc Receptor Ab, and stained with anti-B220-PerCP and anti-Gr1FITC, or anti-B220-PerCP, anti-NK1.1 FITC and anti-DX5 FITC, or anti-B220-PerCP with anti-CD19APC, or anti-B220-PerCP with anti-CD11c APC, or anti-B220-PerCP with anti-CD14 FITC. Isotype-specific controls were performed. Flow cytometric profiles are representative of at least three experiments in C57BL/6J (H-2^{b}), two experiments in C57BL/10 (H-2^{b}) and two experiments in B10.BR/SgSnJ (H-2^{k}). The re-analysis of sorted FC stained with different isotype Abs allowed us to verify the purity of the population (>95%) and the absence of T cell contaminants (<1%).

FIG. 1b: Morphology of sorted CD8⁺/TCR⁻ FC were examined by Wright Giemsa staining with optical microscopy at 2 different magnifications.

FIG. 1c: Morphology of sorted CD8⁺/TCR⁻ FC were examined by transmission electronic microscopy.

FIG. 2a: CD11⁺FC resemble p-preDC. CD11c⁺FC present a p-preDC phenotype. Sorted FC were stained with anti-B220-PercP, anti-CD11c-APC, and anti-CD11b-FITC after blocking. The CD11c^{dim} population (up to 70% of the total FC gate) was analyzed for B220 and CD11b expression. Flow cytometric profiles are representative of at least three separate experiments in both C57BL/6J (H-2^{b}) and C57BL/10 (H-2^{b}).

FIG. 2b: CD4 FC present a p-preDC phenotype. Freshly sorted FC from bone marrow were stained with anti-CD11c FITC, anti-B220 PerCP and anti-CD4 APC Abs after FcR blocking. The CD4⁺ population was analyzed for B220 and CD11b expression. Flow cytometric profiles are representative of at least two separate experiments in C57BL/J6J (H-2^{b}).

FIG. 2c: Morphology of the majority of sorted CD8⁺/TCR⁻ FC were examined after Wright-Giemsa staining under optical microscopy (×100)

FIG. 2d: Morphology of the majority of sorted CD8⁺/TCR⁻ FC were examined by transmission electronic microscopy.

FIG. 3a: FC exhibit in vitro function similar to p-preDC. FC secrete IFN-α. Bone marrow FC and B220⁺/CD11c^{dim}/CD11b⁻ p-preDC were cultured with medium only or CpG. Culture cell free supernatants were collected after 12 hours or 24 hours and IFN-α production was assessed by ELISA. Data are means±s.e.m. of at least two experiments, run in duplicate.

FIG. 3b: FC secrete TNF-α. Bone marrow FC and p-preDC were cultured with medium only or CpG ODN. Culture cell free supernatants were collected after 24 hours and TNF-α was assessed by ELISA. Data are means±s.e.m. of at least two experiments, run in duplicate.

FIG. 3c: FC secrete other pro-inflamatory cytokines. Bone marrow FC (0.05×10⁶ cells/well) were cultured with medium only or CpG ODN. Supernatants were collected after 18 hours and MIP 1□CCL3), MCP-1 (CCL2), RANTES (CCL5), IFN-□, IL-6, IL-10, IL-12p70, and IL-9 were assessed by LINCOplex™ Multiplex Immunoassay. Data are means±s.e.m. of three separate experiments run in duplicate.

FIG. 3d: Upregulation of activation markers on FC. Sorted FC and p-preDC from BMC were cultured with medium (black histograms) or CpG ODN (gray histograms) for 18 hours and stained with the MHC-Class II, CD80 or CD86 FITC-labeled, or isotype control (filled histograms) mAbs. Data on expression of markers are representative of at least four experiments on FC and three experiments on p-preDC.

FIG. 3e: CpG enhances survival of FC. Sorted FC and p-preDC were cultured with medium only or CpG ODN for 18 hours and then stained with 7AAD. Data are means±s.e.m. of dead cells from quadruplicate samples from at least three experiments. The total cell recovery (viable+dead) was 100%. * P=0.024 between p-preDC cultured with medium and with CpG. ** P=0.0038 between FC cultured with medium and with CpG.

FIG. 4a: FL is a key cytokine for FC expansion and maturation in vitro. (a) FC expansion from FL-cultured BMC. Fresh BMC and BMC cultured with FL for 10 days were sorted for CD8α⁺/TCRαβ⁻/TCRγδ⁻ FC, and CD11c⁺CD11b⁻B220⁺ p-preDC. The data shown represents the % of sorted cells in the lymphoid gate and are means±s.e.m. of more than eight experiments for FL-derived FC and FL-derived p-preDC and of more than ten experiments.

FIG. 4b: Morphology of sorted FL-derived FC after 18-hours incubation. Bone marrow FC were sorted from fresh BM or from a 10 day FL-cultured of BM. Cells were incubated for 18 hours with medium or CpG ODN and were examined after Wright-Giemsa staining by optical microscopy at several magnifications. Arrows indicate dendrites.

FIG. 4c: Cytokine secretion of FL-derived FC. Bone marrow FC and p-preDC were sorted from a 10 day FL-cultured of BM and were incubated with medium only or CpG ODN. Culture supernatants were collected after 24 hours and TNF-α, IFN-α and IL12p70 were assessed by ELISA. Data are representative of at least two different experiments, run in duplicate.

FIG. 4d: Upregulation of activation markers on FL-FC. FL-derived FC. FL-derived FC and Fl-derived p-preDC were sorted from a 10 day FL-cultured BMC. FC and p-preDC were also sorted from fresh BM. Cells were incubated for 18 hours with medium or CpG ODN then stained with the MHC-Class II, CD80, CD86 FITC-labeled or isotype control antibodies. Data are means±s.e.m. of FACS analysis of at least three experiments.

FIG. 4e: Viability of bone marrow FL-derived FC after 18-hour culture. Bone marrow FC were sorted from a 10 day FL-cultured of BM or from fresh BM. Cells were cultured for 18 hours with medium or CpG ODN and then stained with 7AAD. Data are means±s.e.m. of viable cells from quadruplicate samples from at least three experiments. * P=0.0032 when comparing medium exposure of fresh FC to FL-derived FC and ** P=0.02155 when comparing FL-derived FC to medium versus CpG exposure.

FIG. 5a: In vivo FL-mobilized-FC facilitate HSC engraftment in allogeneic recipients (a.) FACS analysis of subpopulations in sorted FL-mobilized-FC. CD8α⁺/TCRαβ⁻/TCRγδ⁻ FL-mobilized FC were sorted and stained after FcR blocking. Four-color flow cytometry analysis was performed to characterize distinct subtypes. The CD11c^{dim} and the CD11c^{bright} populations were gated and further analysis for the presence of CD11b and B220 marker expression. The dot plots are representative of two independent experiments in each C57BL/6J (H-2^{b}) and B10.BR/SgSnJ (H-2^{k}) mouse strain.

FIG. 5b: FL-mobilized FC from PB facilitate engraftment of HSC (B10.BR→C57BL/10). C57BL/10 recipient mice were conditioned with 950 cGy TBI and were given 5,000 HSC from untreated B10.BR donors either alone or mixed with 30,000 purified FC from untreated B10.BR BM or from B10.BR FL-treated FC from PB. Survival was followed for up to 6 months.

FIG. 5c: Donor multilineage typing of HSC+FL-FC chimeras (B10.BR→C57BL/B10). T-cell (TCRβ), NK cell (NK1.1), B cell (B220), macrophage (Mac-1) and granulocyte (Gr-1) markers were assessed on donor derived (H-2K^{k}) PBL from recipient mice 3 months after transplantation. Data are representative from one chimera out of 4 performed.

FIG. 5d: Survival of skin grafts in mixed allogeneic chimeras (B10.BR→B10). Donor-specific (B10.BR, n=5) and third-party (BALB/c, n=5) skin grafts were transplanted in mixed allogeneic chimeras 3 months after HSC plus FL-mobilized PB FC transplantation. Date shows skin graft survival over 100 days.

FIG. 6a: P-preDC facilitate HSC engraftment in mismatched recipients. Survival curve of allogeneic recipients transplanted with HSC and p-preDC from BM (C57BL/6J→C3H/HeJ). C3H/HeJ recipient mice were conditioned with 950 cGy TBI and were given 5000 HSC either alone (HSC group) or mixed with 30,000 purified FC HSC+FC group, or with p-preDC (CD11c^{dim} CD11b⁻B220⁺lin⁻) (HSC+p-preDC group) from C57BL/6J mice. Some recipient mice were used as irradiation controls. The cumulative survival percentage of recipients is represented by the Kaplan Meir method, and animals were followed for 6 months. * P=0.0083 between the HSC+FC group and the HSC+p-preDC group, and ** P=0.0076 between the HSC+p-preDC group and the HSC group.

FIG. 6b: (b) Multilineage typing of HSC+p-preDC chimeras (C57BL/6J→C3H/HeJ). T-cell (TCRβ), NK cell (NK1.1), B cell (B220), macrophage (Mac-1) and granulocyte (Gr-1) markers were assessed on donor derived (H-2K^{k}) PBL from recipient mice 3 months after transplantation. Data are representative from one chimera of 6 performed.

FIG. 7a: Flow cytometric analysis of bone marrow cells stained with antibodies to CD8α versus αβ and γδ TCR, with gates for FC and T cells, two and four weeks after GFP⁺ HSC transplantation.

FIG. 7b: Survival of conditioned recipients was calculated using Kaplan-Meier estimates. B10.BR (H2^{k}) recipients transplanted with 10,000 B6 (H2^{b}) HSC alone (●), 10,000 B6 HSC and 30,000 B6 FC (▪), or 10,000 B6 HSC and 30,000 2° GFP⁺ B6 FC (◯) (n>4 per group).

FIG. 7c: Flow cytometric analyses of donor H2^{b+} peripheral blood T cells, B cells, monocytes and granulocytes (n=10). Representative contour plots with enumeration of subsets from B6 HSC+2° GFP⁺ FC recipients are shown.

FIG. 7d: Survival of donor B6 (H2^{b}), or third party BALB/c (H2^{d}) skin grafts on recipients of B6 HSC plus 2° GFP⁺ B6 FC was calculated using Kaplan-Meier estimates. Facilitated HSC engraftment that differed significantly from HSC alone are marked (*=p<0.05).

FIG. 8a: Representative autoradiograms of Southern blotted and probed RT-PCR reactions specific for CD3ε on β-actin-normalized T cell, FC, and thymus cDNA. Control sample lacked cDNA. All RT-PCR analyses were repeated at least twice with similar results.

FIG. 8b: Contour plot illustrating the FC gate.

FIG. 8c: The specificity of the stain in B is demonstrated by a contour plot of bone marrow cells stained with isotype and fluorochrome-matched antibodies.

FIG. 8d: Cell sorting strategy to isolate CD3ε^{hi} versus CD3ε^{lo} FC. Histogram plot depicts FC stained with either anti-CD3ε antibody (solid line), or with a fluorochrome and isotype matched control antibody (dashed line).

FIG. 8e: Histogram plot depicts the post-sort analysis of CD3ε^{hi} FC (solid line) versus CD3ε^{lo} FC (dashed line).

FIG. 8f: RT-PCR analyses specific for CD3ε on β-actin-normalized CD3ε^{hi} and CD3ε^{lo} FC cDNA.

FIG. 8g: Similar analyses for CD3δ, TCRα and TCRβ on β-actin-normalized cDNA from CD3ε^{hi} and CD3ε^{lo} FC. Note the absence of TCR transcript in FC and the presence of CD3ε transcript in CD3ε^{lo} FC.

FIG. 9a: Representative CD8α versus TCR (αβ plus γδ TCR) contour plots from flow cytometric analysis of wild-type B6 demonstrate gates for FC and T cells from bone marrow. The average percentage of bone marrow cells within FC and T cell gates is shown for B6, TCRα^{-/-}, TCRβ^{-/-}, CD3ε-tg, CD3ε^{Δ-/Δ-} and CD3δ^{-/-} B6 mouse bone marrow cells (+/−standard error of the mean).

FIG. 9b: Long-term survival of conditioned recipients was calculated using Kaplan-Meier estimates. B10.BR recipients were transplanted with 10,000 B6 HSC alone (●), 10,000 B6 HSC and 30,000 B6 FC (▪), or 10,000 B6 HSC and 30,000 CD3ε-tg FC (▴), 10,000 B6 HSC and 30,000 CD3ε^{Δ-/Δ-} FC (♦), or 10,000 B6 HSC and 30,000 CD3δ^{-/-} FC (⋄), 10,000 B6 HSC and 30,000 B6 TCRβ^{-/-} FC (Δ), or 10,000 B6 HSC and 30,000 B6 TCRα^{-/-} FC (◯), (n≧11 per group). Facilitated HSC engraftment that differed significantly from recipients of HSC alone are marked (*=P<0.05).

FIG. 10: Long-term survival of transplanted syngeneic recipients was calculated using Kaplan-Meier estimates. B6 recipients were transplanted with 1,000 B6 HSC alone (◯), 500 B6 HSC (●), 500 B6 HSC and 30,000 B6 FC (▪), 500 B6 HSC and 30,000 B6 T cells (Δ) (n≧4 per group). Cohorts that differed significantly from recipients of suboptimal numbers of HSC alone are marked (*=P<0.05).

### DETAILED DESCRIPTION OF THE INVENTION

HSC chimerism has the potential to induce tolerance to organ transplants and cure autoimmune diseases. However, the widespread application of this promising therapy in the clinic is incumbent upon reducing the toxicity associated with conventional BMT. Accordingly, a great deal of attention has been focused on identification of cells with facilitating potential. CD8^{+/}TCR⁻ FC were reported to enhance engraftment of purified allogeneic HSC without causing GVHD⁸⁻¹⁰. Until now, the precise characterization of FC has remained controversial due to the heterogeneity of the CD8⁺/TCR⁻ population and the infrequency of the various components. The present invention demonstrates for the first time that a cell subtype which is B220⁺/CD11c^{dim}/CD11b⁻ with a plasmacytoid morphology (p-preDC) is the major component of the CD8⁺/TCR⁻ facilitating cell population, making them likely candidates for the biologic function of facilitation.

The present invention demonstrates for the first time that p-preDC facilitate HSC engraftment in allogeneic recipients. Previously, the heterogeneous CD8⁺/TCR⁻ population was described as sharing phenotypic characteristics with CD8α lymphoid dendritic cells⁸. At that time, the phenotype for murine p-preDC was unknown, making an assessment of the relative contribution of this DC subset to facilitation impossible. The present invention now demonstrates that the B220⁺/CD11c^{dim}/CD11b⁻ cells in the FC gate exhibit a morphology and a phenotype that closely resembles mouse p-preDC. Unlike mouse bone marrow p-preDC, most of which are CD8α⁻, all of the B220⁺/CD11c^{dim}/CD11b⁻ FC express the CD8αantigen. CD8α expression on mouse p-preDC has been demonstrated to vary according to tissue source and state of activation^{32, 36}. CD8αexpression is significantly up-regulated from 10%-30% on resting bone marrow p-preDC to 70-100% after activation²⁶. The inventor, therefore hypothesized that the B220⁺/CD11c^{dim+}/CD11b⁻ FC in the bone marrow represent the 10-30% “resting” BM CD8α⁺p-preDC. This hypothesis is reinforced by the fact that isolated FC from fresh/untreated BM present a low expression of costimulatory molecules (i.e., MHC Class II, CD80, CD86), CD40 (data not shown) and no CD205 expression (data not shown) as has been reported for p-preDC^{26, 37}. Although other cells are also present in the FC population, including B cells, and few NK cells, granulocytes or monocytes, the paucity of these cells in the functional FL-mobilized PB-FC population (manuscript in preparation) leads us to conclude that these cells do not play a significant role in facilitation and reinforces the hypothesis that DC, representing over 80-90% of the mobilized FC population with a predominance of p-preDC, are central to facilitation.

The present invention shows that FC share many features with p-preDC, including their response to CpG ODN with: 1) secretion of similar cytokines and chemokines, 2) maturation and, 3) improved survival in culture. The hallmark of p-preDC is the capacity to produce high amounts of IFN-type I, consisting of IFN-α, IFN-β, and IFN-ω, in response to appropriate stimulation^{19, 25}. Mouse p-preDC, as is true for their human counterparts, respond preferentially to ligands for TLR7 and TLR9 and only poorly to ligands for TLR2, TLR3 or TLR4³⁸. Notably, FC produce IFN-α after stimulation with CpG ODN, and none after stimulation with LPS (TLR4 ligand) (data not shown). Besides IFN-α, FC produce pro-inflammatory cytokines and chemokines, including MIP-1-α, MCP-1, TNF-α, RANTES, IL-6, IFN-γ and IL-12p70. Therefore, FC, like p-preDC, appear to preferentially produce proinflammatory cytokines and chemokines³⁹ that could lead to the induction of a Th-1 type immune response. These data are not contradictory with the concept of “tolerogeneic” DC, since p-preDC are potently tolerogeneic under selected circumstances. Namely, p-preDC have been shown to induce anergy in an antigen-specific CD4⁺ T cell line⁴⁰; differentiation of naive CD4 and CD8 T-cells into Th2 cells⁴¹; and T regulatory cell differentiation⁴². In light of the similarities between pre-DC and FC, it is possible that FC induce immune deviation to promote a tolerogeneic milieu for HSC engraftment either via cytokines and/or generation of regulatory T cells. The fact that FC produce IL-10, a potent anti-inflammatory cytokine⁴³ that is used to generate regulatory T cells in vitro or in vivo^{44, 45} supports this hypothesis that the generation of regulatory T cells after FC transplantation may enhance engraftment by tolerizing alloreactive responses. Interestingly, FC highly upregulate CD86 expression after CpG ODN stimulation. It is therefore possible that after transplantation, the CD86 on FC interacts with its ligand, CTLA-4, on T cells, leading to a decrease in allogeneic T cell responses.

In humans and in mice, the development of p-preDC is dependent upon FL³⁴. Similarly, FL is also a key cytokine for FC generation and expansion, as evidenced by the FL-BM cultures and the mobilization of FC in PB³⁵. FL-treatment in vivo induces the maturation/activation of FC, demonstrated by the presence of 20% mature lymphoid DC (B220⁻CD11c^{bright+}CD11b⁻) that express CD86. Similarly, FC propagated from BMC in vitro exhibit presence of dendrites and upregulation of activation markers. The present invention demonstrates that purified FL-mobilized FC facilitate HSC engraftment very efficiently. The mobilization of FC by FL could represent a more efficient approach to recruit functional “facilitating” or “tolerogeneic” cells for clinical application when limited numbers of cells are available for transplantation.

To date, there has been only indirect evidence to demonstrate a sustained tolerogenic effect for p-preDC in vivo. The present invention shows for the first time that p-preDC exhibit a significant graft-enhancing ability in mismatched recipients. Notably, p-preDC significantly enhanced engraftment of HSC without causing GVHD. Therefore, the tolerogenic effect of this cell population was maintained in vivo as it relates to establishing chimerism and tolerance.

It is interesting that the total FC population exhibits a significantly engraftment-enhancing effect on HSC than p-preDC. A number of hypotheses could explain this observation. P-preDC-like cells in the FC gate, may be the rare CD8α⁺ subpopulation of the total p-preDC found in the bone marrow, and only CD8α⁺ p-preDC may be able to fully replace FC in this functional biological assay. P-preDC may also not be in an appropriately activated state. Given the heterogeneous nature of cells in the FC gate, it is possible that another collaborative cell population (i.e. NK cells) is required for optimal function of p-preDC. In support of this mechanism is the fact that activation of p-preDC by NK cells in vitro has been reported⁴⁶. It is also possible that p-preDC-phenotype cells within the FC gate are distinct from the bulk population of bone marrow p-preDC (other than activation or known maturation status).

In conclusion, CD8⁺/TCR⁻ facilitating cells will have a significant impact for the clinical application of HSC-induced chimerism since tolerance can be promoted, GVHD avoided, and safe transplants allowed in mismatched recipients^{10, 47, 48}. Notably, the present invention demonstrates for the first time in vivo effect for p-preDC in facilitating HSC engraftment and inducing durable tolerance to transplanted grafts but with less efficiency than FC. The identification of the cells in the FC gate and the mechanism by which they mediate a full facilitation of HSC engraftment will lead to novel cell-based therapeutic strategies to optimize the composition of the graft in order to reduce the morbidity of HSC transplants in mismatched recipients.

Both recipient and donor conditioning methods are well known in the art. The present invention is directed toward augmenting a conditioning method by treating the donor or recipient with FL. Flt3 ligand may be used for purposes of mobilization by administering 1 g/kg-30 g/kg per day for 1-15 days. Preferably, FL is administered at 15 g/kg-25 g/kg per day for 5/15 days, or 20 g/kg for about 10 days.

The Flt3 ligand (FL) disclosed in the method of the present invention can be administered to a patient by any available and effective delivery system including, but not limited to, parenteral, transdermal, intranasal, sublingual, transmucosal, intra-arterial, or intradermal modes of administration in dosage unit formulations containing conventional nontoxic pharmaceutically acceptable carriers, adjuvants, and vehicles as desired, such as a depot or a controlled release formulation.

For example, a pharmaceutically acceptable formulation of the composition of the present invention may be formulated for parenteral administration, e.g., for intravenous, subcutaneous, or intramuscular injection. For an injectable formulation, a dose of the composition of the present invention may be combined with a sterile aqueous solution which is preferably isotonic with the blood of the patient. Such a formulation may be prepared by dissolving a solid active ingredient in water containing physiologically-compatible substances such as sodium chloride, glycine, and the like, and having a buffered pH compatible with physiological conditions so as to produce an aqueous solution, and then rendering the solution sterile by methods known in the art. The formulations may be present in unit or multi-dose containers, such as sealed ampules or vials. The formulation may be delivered by any mode of injection, including, without limitation, epifascial, intracutaneous, intramuscular, intravascular, intravenous, parenchymatous, subcutaneous, oral or nasal preparations (see, for example, U.S. Pat. No. 5,958,877, which is specifically incorporated herein by reference).

HSC chimerism has the potential to cure a number of disease states. However, its widespread application is limited by the toxicity of GVHD when unmodified marrow is transplanted. As a result, the search for cells with facilitative function has been pursued. Both CD8⁺/TCR⁺ and CD8⁺/TCR⁻ bone marrow cells facilitate HSC engraftment in allogeneic recipients [8, 9, 48]. A clear definition of the mechanism by which these populations facilitate has not been reported. In the present invention, FC were biologically separated from T cells and the potential mechanism of action for facilitation in syngeneic as well as allogeneic recipients was elucidated. The data presented herein shed light on conflicting reports further characterizing CD8⁺/TCR⁻ and other facilitating cell subpopulations [8, 48, 55]. In the model of Gandy et al., which used only 800 cGy TBI (vs. 950), both CD8⁺/TCR⁻ and CD8⁺/TCR⁻ FC were required for the full facilitating effect. In light of the fact that with full ablation CD8⁺/TCR⁺ cells cannot substitute for CD8⁺/TCR⁻ FC, and are not required for FC function, it is highly likely that the CD8⁺/TCR⁺ subpopulation of conventional T cells was required to veto the radioresistant cells in the hosts conditioned by 800 cGy TBI. The data presented herein using a more ablative conditioning model therefore clearly separate CD8⁺/TCR⁻ FC from conventional CD8⁺ T cells.

In this model, several late event deaths occurred. These data were interpreted as a failure of long term engraftment of the self-renewing HSC. It is possible that a decrease in self-renewal would lead to exhaustion of long-term repopulating HSC over time. These mice would probably die sooner in less strict barrier environments. However, infection as a possible mechanism of death can be excluded, because the colony was screened monthly for 15 pathogens, and was run as a strict barrier facility. Instead, the tolerance induced by the transplant may not have been complete, leading to late rejection and death.

The molecular analyses presented herein have clearly distinguished FC from T cells. Although both FC and T cell subsets express the transcript for CD3ε, suggesting a common ontogeny, FC do not express TCR gene transcripts which are expressed by conventional T lymphocytes. Specifically, RT-PCR analyses of double-sorted FC revealed a lack of signal for TCRα, as well as TCRβ. Moreover, the generation of FC does not require genes encoding the T cell receptor as evidenced by the fact that FC from TCRα^{-/-} donors facilitate HSC engraftment as well as normal controls. Therefore, it is highly unlikely that the facilitating effect is due to contaminating αβ T cells, since mice without αβ T cells (TCRα^{-/-}) produce functional FC (Table 2).

In agreement with a previous report [48], the inventor found that TCRβ^{-/-} FC do not facilitate. However, the CD8⁺/TCR⁻ FC population analyzed according to the present invention by RT-PCR does not contain transcripts for TCR genes. Schuchert et al. did not look for TCRβ transcripts, but identified TCRβ protein on FC using a monoclonal antibody [48] that may be cross-reactive with an unknown receptor component. For a genetic mutation to cause a cell autonomous defect, the affected cell must express the gene. A defect in a cell that does not express the gene is likely to be indirectly affected by a mutation. Specifically, defects in cells that do express the gene may affect other cells. Thus, a mutant phenotype in FC without expression of the TCRβ gene in FC suggests that the defect engendered by deletion of TCRβ is not an intrinsic or cell autonomous defect. Instead, changes in other as yet unknown populations may affect FC in the TCRβ^{-/-} mutant mice. This discrepancy is highlighted by the fact that TCRα is not expressed in FC and mice without TCRα produce functional FC. In stark contrast, multiple components of the CD3 complex are expressed in FC, and all CD3 mutant mice examined demonstrate defective FC activity. These findings therefore suggest that TCR genes, such as TCRα and TCRβ, are not absolutely required for FC function.

The data presented herein reveal a critical requirement for CD3ε in FC facilitation of allogeneic HSC engraftment. These data resemble that for mature T cells which also critically require CD3ε for CD3 complex signaling, activation and function [60]. Both the expression of the CD3ε-transgene and deletion of CD3ε in CD3ε^{Δ-/Δ-} mice have been shown to disrupt CD3 complex assembly and ablate T lymphopoiesis [60, 62]. As in T cells, function of CD3ε-tg FC and CD3ε^{Δ-/Δ-} FC is significantly impaired in allogeneic recipients. It is therefore possible that the development of the CD3 complex is prerequisite for production of functional FC. Indeed, it would appear that a CD3 complex is formed in CD3ε^{hi} FC, since CD3δ is also expressed in CD3ε^{hi} FC, but not CD3ε^{lo} FC. Importantly, deletion of CD3δ impairs FC function. Similarly, deletion of CD3δ is associated with impaired T cell function [61]. Taken together, the data presented herein strongly suggest that the CD3 complex may play a role in signaling within FC. It is hypothesized that the CD3ε^{hi} FC which contain the transcript for CD3δ represent a more mature or functionally activated form of FC.

The critical requirement for CD3ε in FC-mediated HSC engraftment in allogeneic recipients could be explained by a number of hypotheses. It is possible that CD3ε may be required for development of the specific receptor on FC. Schuchert et al. demonstrated that FC possess a CD3ε-containing cell surface receptor complex (FCp33), and hypothesized that the complex may be involved in MHC recognition [48]. The findings presented herein support the general hypothesis that a CD3 complex-containing receptor on FC mediates MHC recognition. Notably, FC and HSC must be MHC matched for facilitation of HSC engraftment into a recipient that is not genetically MHC matched to either cell donor [9, 48, 63]. Specifically, in the absence of MHC matching between HSC and recipient, FC congenic to HSC only at class I K allow facilitated engraftment in MHC-disparate recipients [49]. These data may point to the need for MHC recognition by FC. CD3ε should be intrinsically required by FC to mediate some functional aspect of allogeneic HSC engraftment. It is likely that CD3 proteins would mediate signaling from the FCp33 complex during allorecognition. Without CD3 components, signaling from the proposed complex would be defective. Subsequently, these dysfunctional FC may interfere with HSC engraftment by attempting to perform their function without the ability to signal and activate. Indeed, the data from CD3δ^{-/-} mice may be reconsidered in light of the fact that deletion of CD3δ generates a specific TCR signaling defect, and CD3δ^{-/-} FC are impaired in function. One could hypothesize that similar altered signaling might also be found in the FCp33 complex.

Loss of CD3ε results in a severe impairment to HSC engraftment, whereas the engraftment of HSC is not affected positively or negatively by FC devoid of CD3δ. Since FC express transcripts for CD3ε and CD3δ, it is more likely that these defects are cell autonomous or intrinsic to FC. Interestingly, CD3ε is more critically required for T cell signaling than CD3δ [60-62]. Likewise, the T cell defects engendered by deletion of CD3ε are more profound than those imposed by CD3δ mutation [60-62]. It is therefore possible that signaling and activation in CD3ε^{-/-} FC, and subsequent effects on HSC engraftment, are more severely altered than in CD3δ^{-/-} FC.

An alternative explanation for the requirement for CD3ε might be that other cells that mediate a functional maturation of FC may require CD3ε to develop or function. Without CD3ε, such helper cells would be absent or impaired, and FC would remain functionally immature. One would also have to hypothesize that the reason why CD3ε^{Δ-/Δ-} FC fail to facilitate HSC in allogeneic recipients is that they are immature. Gandy et al. indicated that FC display some cell surface markers compatible with CD8α⁺ dendritic cells (DC) [8]. Indeed, culturing early CD8⁺ thymocyte precursors under conditions permissive for DC development induces both CD3ε and CD3δ in the DC [64]. These data set a precedent for the expression of CD3ε and CD3δ transcripts in non-T lineage cells like the FC In addition, a defect in IL-12 production by CD3ε^{Δ-/Δ-} DC was relieved by adoptive transfer of T lymphocytes, or co-culture of DC with T lymphocytes [64]. Thus, in a manner similar to CD3ε^{Δ-/Δ-} DC which function in some assays but do not produce IL-12, CD3-mutant FC may lack some critical cytokine or other priming required to facilitate in allogeneic recipients. Following this line of reasoning, the level and type of T cells in a mouse should then determine FC functionality. The data presented herein show that TCRα^{-/-} mice generate functional FC. While TCRα^{-/-} mice do not produce αβ T cells, they do make a few ββ T lymphocytes [65], and these T cells may be enough to affect FC function. While this theory is attractive, it is difficult to reconcile with the data presented herein from the significantly impaired function of FC from CD3δ^{-/-} mice. While CD3δ^{-/-} mice have a 30-fold reduction in αε T cells [61], a low level of αβ T cells is produced. If FC function in the absence of T lymphocytes (Table 2), then T cells are not absolutely required to produce mature FC. The data presented herein do not exclude the possibility that an as yet unknown cellular population that critically requires CD3ε for development or function is required for maturation of functional FC.

The fact that FC are capable of facilitating limiting numbers of syngeneic HSC is a new demonstration of the unique functional capacity of these cells. T cells do not substitute for FC in this assay. In a recent report, T lymphocytes were shown to improve HSC homing and short term engraftment [66]; however, as in the present invention, the inclusion of T cells with HSC in a syngeneic recipient did not lead to long-term HSC engraftment in vivo. FC must therefore act to mediate HSC engraftment by mechanisms beyond those used by T cells, such as removing host alloreactivity, or increasing the efficiency of HSC homing. The inclusion of FC in marrow grafts may be critical for HSC engraftment in clinical situations where HSC numbers are limiting. Moreover, the present data confirm that CD8⁺/TCR⁺ T cells are not essential to FC function or facilitation. As the role for myelotoxic conditioning is defined, cell-based strategies to induce host-versus-graft tolerance and increase the efficiency of engraftment will significantly reduce the morbidity associated with conventional BMT.

In order to illustrate the invention, the following examples are included. However, it is to be understood that these examples do not limit the invention and are only meant to suggest a method of practicing the invention. Persons skilled in the art will recognize that non-exemplified methods may be successfully performed by making routine modifications apparent to those skilled in the art.

### EXAMPLES

Material and Methods

Mice. 5-10 week-old male B10.BR/SgSnJ (H-2k), C3H/HeJ (H-2k), C57BL/10SnJ (H-2b), BALB/c (H-2d), or C57BL/6J (H-2b) mice were purchased from Jackson Laboratories (Bar Harbor, Me.). Rodents were maintained under pathogen-free conditions in the animal care facility at the Institute for Cellular Therapeutics, according to specific University of Louisville, Institutional Animal Care and Use Committee and National Institutes of Health animal care guidelines.

Flt3-ligand treatment of mice. Recombinant human Flt-3 Ligand (FL, kindly provided by Amgen, Seattle, Wash.) was diluted in sterile, filtered, endotoxin-free water at a concentration of 100 μg/ml. Donor B10.BR mice were subcutaneously injected with 10 μg of FL daily for 10 days. B10.BR control mice were injected with saline only. At the end of the treatment, peripheral blood was harvested and collected into heparinized tubes.

mAbs and Flow cytometry. The following mAbs (all from BD Biosciences Pharmingen, San Diego, Calif., except those labeled with APC-cy-7 from eBioscience, San Diego, Calif.) were used. To sort CD8⁺/TCR⁻ FC: anti-CD8α (53-6.7) PE-labeled, anti-TCR β chain (H57-597) and anti-TCR γδ chain (GL3) FITC-labeled. To sort B220⁺/CD11c^{dim}/CD11b⁻ p-preDC: anti-CD11b (M1/70) APC-labeled, anti-CD45R/B220 (RA3-6B2) APC-Cy7-labeled, anti-CD11c (HL3) PE-labeled, and anti-TCR β chain (H57-597), anti-TCR γδ chain (GL3), anti-CD14 (rmC5-3), anti-CD19 (1D3), anti-Pan-NK cells (DX5), and anti-NK1.1 (PK136), all FITC-labeled. To sort HSC: anti-TCR β chain (H57-597), anti-TCR γδ chain (GL3), anti-Ly-6G (Gr-1), anti-CD11b, Mac1 (M1/70), anti-CD8α (53-6.7) and anti-CD45R/B220 (RA3-6B2), all FITC-labeled, anti-Ly-6A/E (Sca-1) (E13-161.7) PE-labeled and anti-c-Kit (CD117) (2B8) APC-labeled. To restain the sorted FC population: purified anti-CD16/CD32 (FcγIII/II receptors) (2.4G2), anti-CD11c (HL3), anti-CD4 (RM4-5) and anti-CD19 (1D3) all APC-labeled, anti-CD45R/B220 (RA3-6B2) PerCP-labeled, and anti-Pan-NK cells (DX5), anti-NK1.1 (PK136), anti-CD14 (rmC5-3), and anti-Ly-6G (Gr-1) FITC-labeled. To analyze the activation state: anti-CD80 (B7-1) (16-10A1), anti-CD86 (B7-2) (16-10A1), and anti-I-A^{b} (Aβ^{b}) (AF6-120.1) FITC-labeled. For PBL typing and multilineage chimerism: anti-H-2 K^{b} (AF6-88.5) PE or FITC-labeled, anti-H-2K^{k} (36-7-5) PE or FITC-labeled, anti-TCR β chain (H57-597), anti-NK1.1 (PK136), anti-Ly-6G (Gr-1), anti-CD11b, Mac1 (M1/70) and anti-CD45R/B220 (RA3-6B2) all FITC-labeled. Detection of dead cells after cell culture was determined by using a 7-Amino-actinomycin D (7-AAD) (Molecular Probes, Eugene, Oreg.) followed by FACS analysis.

Bone marrow cell (BMC) preparation. BMC preparations were performed as previously described⁹. Briefly, BMC were obtained by flushing femurs and tibias from mice with cold Media 199 (Gibco, New York, N.Y.) containing 30 μg/ml Gentamicin (Gibco) (referred to hereafter as chimera media, CM). After washing with CM, the BMC were resuspended to 100×10⁶ cells/mL in sterile Cell Sort Media (CSM: Hank's Balanced Salt Solution without phenol red (Gibco), 2% heat inactivated fetal calf serum (Gibco), 2 kg/mL hepes buffer (Gibco) and 30 μg/ml of Gentamicin (Gibco)).

Culture of BMC with FL. BMC were resuspended at 10⁶ cells/ml in culture medium consisting of RPMI 1640 (Gibco), 10% FBS (Gibco), 1 mM Sodium pyruvate (Gibco), 10 mM Hepes (Gibco), 2 mM L-Glutamine (Gibco), Penicillin 100 U/mL, 100 μg/mL Streptomycin, (Gibco), and 10⁻⁵ M 2-mercaptoethanol (Sigma), supplemented with human FL (100 ng/ml, generous gift from Amgen, Seattle, Wash.). Every 5 days of culture, half of the medium was replaced by fresh cytokine-supplemented culture medium according to a protocol previously described³⁰.

Cell sorting. HSC, FC, and p-preDC were sorted as previously described^{9,30,49}. HSC were sorted for Sca-1⁺c-Kit⁺Lin⁻ expression, FC were sorted for CD8α⁺/TCRαβ⁻/TCRγδ⁻ expression, and p-preDC were sorted for CD11c^{dim}/CD11b⁻/B220⁺ expression. Briefly, BMC were incubated with Abs on ice for 30 minutes, cells were washed twice in the CSM, filtered, and resuspended to a final concentration of 2.5×10⁶ cells/mL in the CSM for cell sorting. The isolation of the cell populations was performed on FACSVantage Flow Cytometers (Becton Dickinson). The populations of interest were isolated from the live lymphoid gate, and after reanalysis, only cells with a purity of >94% were used.

Phenotypic analysis of sorted FC. Sorted FC isolated from fresh BMC or FL-mobilized peripheral blood, with a purity ranging from 94 to 98%, were incubated with Fc receptor block (anti-CD16/CD32) before staining with different lineage specific markers, including anti-CD4, CD11c, B220, NK1.1, DX5, CD14, Gr1, and CD19. During reanalysis of sorted FC, less than 1% of contaminating T cells (TCRαβ FITC and TCRγδFITC) were detected, which therefore allowed the use of FITC-conjugated specific markers for further analysis. To validate the specificity of staining, various conjugates of the same antibody were used, including CD11c, CD4 and CD19 conjugated to FITC and APC, as well as B220 conjugated with PerCP, APC, or FITC (not shown). To analyze the subtypes of DC in the sorted FC population, sorted FC were blocked and stained with CD11b FITC, CD11c APC and B220 PerCP mAbs to determine the presence of myeloid pre-DC (CD11c⁺/B220⁻/CD11b⁺), p-preDC (CD11c^{dim}/B220⁺/CD11b⁻), the common precursor population for DC (CD11c⁺/B220⁺/CD11b⁺) or mature lymphoid DC (CD8α⁺/CD11c⁺/CD11b⁻/B220⁻). The cells were washed twice in CSM, and analyzed on a FACS Calibur using Cell Quest Software (Becton Dickinson, Mountainview, Calif.).

CpG oligodeoxynucleotides (ODN) stimulation. Sorted p-preDC or FC were cultured for 18 hours at 10⁵ cells/200 μL in 96 well round-bottom culture plates in culture medium in the presence or absence of Toll-like receptor (TLR)-9 ligand, CPG-ODN 1668 (TCCATGACGTTCCGATGCT) (SEQ ID NO. 1) (GibcoBRL Custom Primers) at 1 μM, TLR-4 ligand, LPS from Escherichia coli (Sigma, MO, USA) at 10 μg/ml, as previously described³⁰. CpG or LPS-treated or untreated cells were subsequently assayed for: 1) the expression of DC activation/maturation cell surface markers by FACS, 2) their survival rate by 7AAD staining, or 3) morphological appearance by Wright Giemsa staining on cytospins. The supernatant of these cultures were collected for analysis of the production of different cytokines by ELISA.

Cytospins. Cells (30,000 to 60,000) were centrifugated for 5 minutes at 300 rpm. The slides were air dried, fixed with methanol and dried at room temperature. Wright Giemsa staining was performed using the kit Hema3 according to the manufacturer's protocol (Fisher, PA, CA).

Transmission electron microscopy. Cells were pelleted at 1,000 g, fixed in situ as a pellet in 2.5% glutaraldehyde, and processed for transmission electron microscopy using standard methods. Sections of 70 nm were cut with a Reichert Ultracert S mounted on copper grids and counterstained with uranyl-acetate (2%) and lead citrate. Observations were performed using a Joel 100 cx electron microscope.

Cytokine production by ELISA. Briefly, the cell-free supernatants of 12H, 18H, or 24H cultured cells (FC or p-preDC,) with or without CpG ODN or LPS were collected and kept frozen at −80° C. The amount of cytokine produced was determined by 1) ELISA kits for mouse IFN-α (R&D system) and mouse TNF-α (Biosource International), and 2). Multiplex for MIP 1αCCL3), GM-CSF, MCP-1 (CCL2), RANTES (CCL5), IFN-γ, IL-1β, IL-2, IL-4, IL-5, IL-6, IL-10, IL-12p70, IL-9, and IL13 on 18 hour incubation supernatants from three different experiments performed by Linco Diagnostic Services (St. Charles, Mo.).

Reconstitution of allogeneic recipients with HSC from untreated marrow and FL-mobilized FC. HSC were sorted from untreated B10.BR mice (H-2^{k}). FC (CD8⁺/TCR⁻) were sorted from PB of 10 day FL-treated B10.BR mice, or from untreated B10.BR mice as controls. Recipient C57BL/10SnJ mice (H-2^{b}) were treated with 950 cGy of total body irradiation (TBI) using a 137-cesium source (Gamma-cell 40 Excutor, Nordion International, Ontario, Canada). Four to six hours after irradiation, 5,000 HSC were transplanted either alone or in combination with 30,000 FC by lateral tail vein injection into C57BL/10SnJ recipients. FC and HSC were mixed prior to transplantation. A control group of irradiated mice was also established. The survival was plotted over time.

Reconstitution of allogeneic recipients with HSC +/− bone marrow FC or p-preDC. Recipient mice (C3H/HeJ, H-2^{k}) were given 950 cGy TBI. Six hours after irradiation, recipients were transplanted with 5,000 purified allogeneic HSC (C57BL/6J, H-2^{b}) with or without 30,000 FC or p-preDC resuspended in CM, via lateral tail vein injection. A group of irradiated mice served as controls. Graft survival was estimated according to the Kaplan-Meier method.

Characterization of chimeras and donor multilineage engraftment by flow cytometry. Donor engraftment in the recipient was quantified by peripheral blood cell typing using flow cytometry. Specifically, two-color flow was used to determine the percentage of PBL that express H-2^{b} or H-2^{k} MHC class I antigen. Briefly, whole blood from recipients was collected into heparinized tubes, and aliquots of 100 μL were stained with anti-H-2K^{b}-FITC and anti-H-2K^{k}-PE. Red blood cells were lysed with ammonium chloride lysing buffer for 5 min at room temperature, and the samples were then washed twice in FACS medium (Hanks balanced salt solution, (Gibco), sodium bicarbonate (Sigma), bovine serum albumin (Sigma) and sodium azide (Sigma)) and either analyzed fresh using a FACSCalibur or fixed in 1% formaldehyde (Polysciences, Warrington, Pa.). For multilineage analysis, PBL were stained with donor-specific anti-H-2K^{b}-PE or anti-H-2K^{k}-PE mAb along with a combination of the following Abs: anti-Gr-1, anti-Mac-1, anti-αβ-TCR, anti-B220, anti-NK1.1, anti-CD11c and anti-CD19. Cells were washed, acquired and analyzed on the FACS Calibur.

Skin grafts. Skin grafts were performed by techniques published previously⁵⁰. Briefly, full-thickness skin grafts from the tail of B10.BR, B10, C57BL/6J or BALB/c mice were harvested. Full-thickness graft beds were prepared on the lateral thoracic wall. Three skin grafts (syngeneic, donor, and third party) were placed on each animal. Each graft was separated from the others by a skin bridge of at least 3 mm. Skin grafts were covered by a double layer of petroleum gauze and a cast. The cast was removed after 7 days. Grafts were scored daily for percent rejection. Rejection was defined as complete when no residual viable graft could be detected.

Significance estimates. Mice survival was estimated according to the Kaplan-Meier method and tested with the log rank statistic. The cumulative survival estimates the percentage of mice alive after a given amount of time. The graph was plotted according to days after transplantation versus cumulative survival percentage. Transplantation experiments were started at different time points and the mice were censored after different amounts of time. All graphs of transplanted mice represent experimental animals from at least three separate days of sorting and transplantation. For the other experiments, statistical significance was determined by application of the Student's T-test; p<0.05 was considered significant.

### Example 1

### CD11c⁺ Cells are the Predominant Cell Type within the FC Population

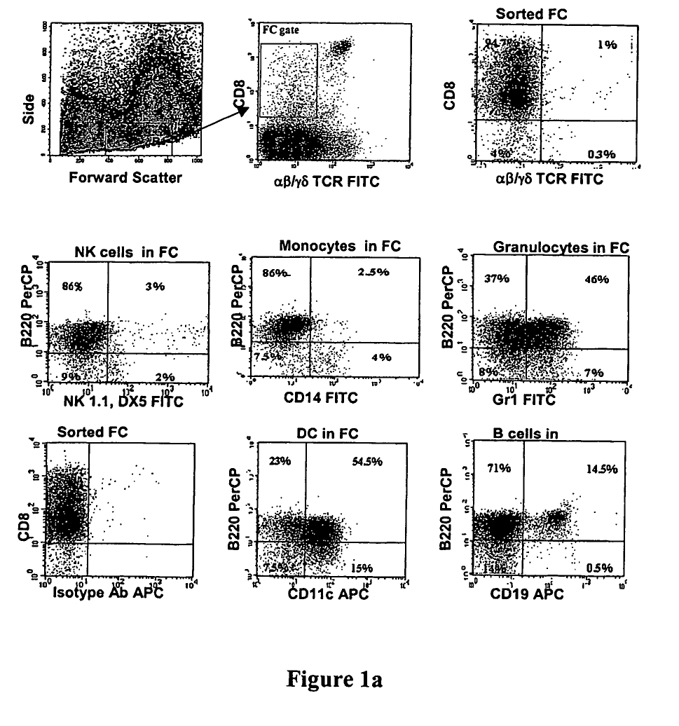

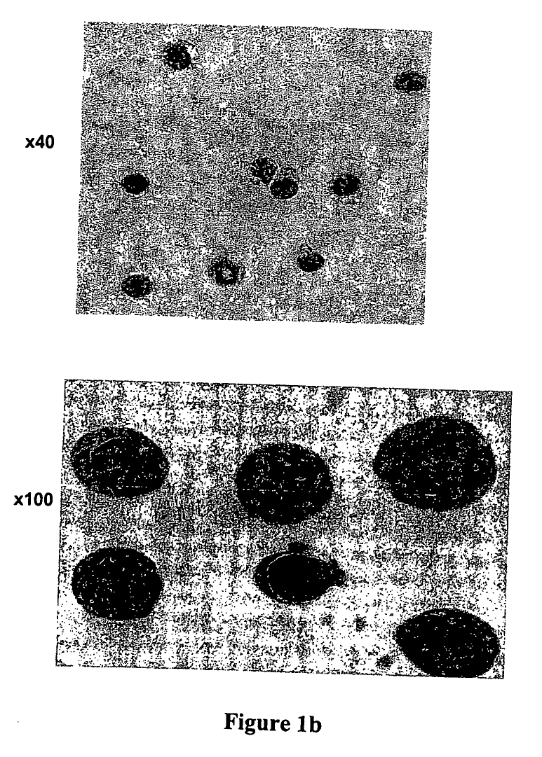

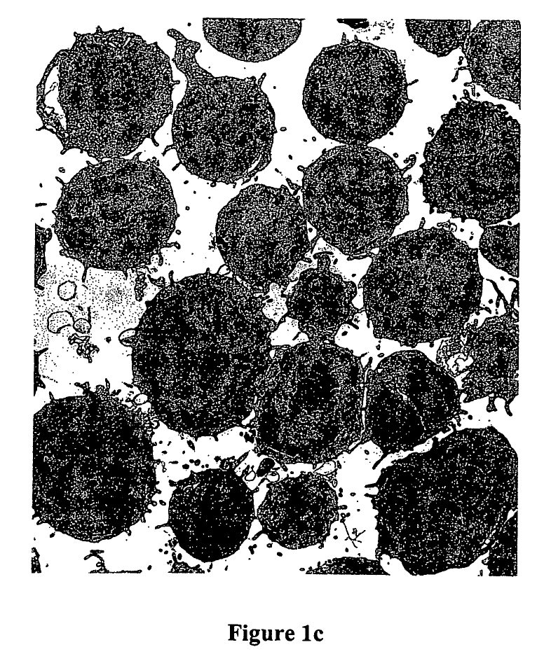

To better characterize FC, markers expressed on the sorted CD8α⁺/TCR⁻ FC population were analyzed by FACS analysis. Approximately 65-70% of FC express CD11c⁺ (FIG. 1a), and 75-88% of FC express B220 (FIG. 1a). Among the subpopulations negative for B220 expression, approximately 4-6% were NK (NK1.1⁺ and DX5⁺), 6-7% were granulocytes (Gr1⁺) and 2-4% were monocytes (CD14⁺) (FIG. 1a). Among the B220⁺ subpopulation, only 15% were B cells (CD19⁺) (FIG. 1a), and 65% were DC (CD11c⁺). The CD19⁺B220⁺FC subpopulation was also positive for intra-cytoplasmic IgM (data not shown), confirming a B cell phenotype. Taken together, these data demonstrate that there are distinct subpopulations within the sorted FC that include a minority as NK, granulocytes, monocytes, B cells and a majority as DC. In addition, the sorted FC exhibited a variety of morphologies representing different cell types on cytospins with Wright-Giemsa staining (FIG. 1b). The heterogeneity of the sorted FC was further confirmed by transmission electronic microscopy (FIG. 1c).

### Example 2

### 95% of CD11c⁺ FC Resemble Plasmacytoid Precursor DCs (p-preDCs)

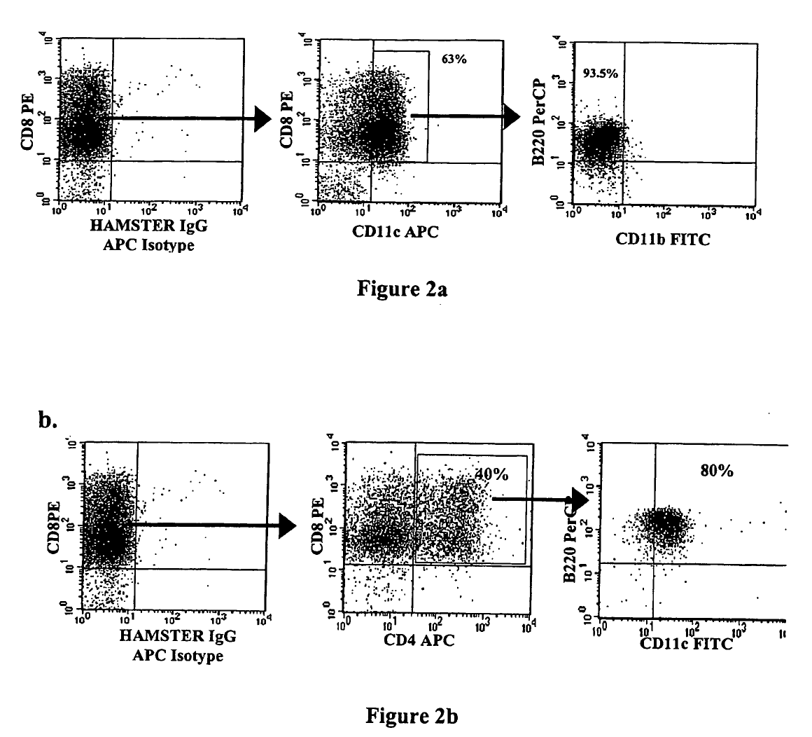

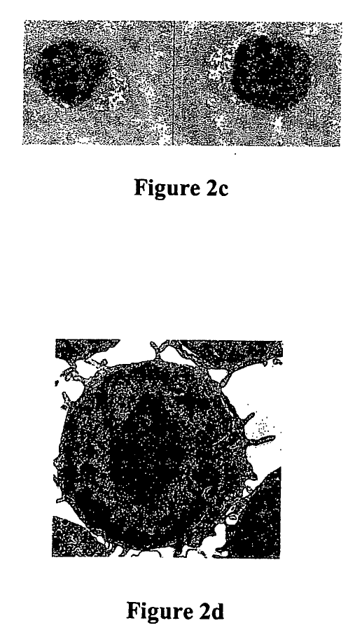

Because CD11c⁺ DC represent the largest subset in the FC (up to 70%), the known subtypes of DC present in the sorted FC population were analyzed by FACS analysis (FIG. 2a). Strikingly, p-preDC (CD11c^{dim}/B220⁺/CD11b⁻) comprised 93-95% of the CD11c⁺FC subpopulation (FIG. 2a). To confirm that the predominant CD11c⁺FC subpopulation was related phenotypically to p-preDC, the presence of the CD4 marker on sorted FC was analyzed, at least 70% of bone marrow p-preDC has been shown to express the CD4 antigen²⁶. Approximately 40-50% of FC expressed CD4 (FIG. 2.b) and this CD4⁺ FC subpopulation was almost exclusively of plasmacytoid phenotype (CD11c⁺B220⁺). Further, the majority of cells in the sorted FC population not only presented a p-preDC cell surface phenotype, but also exhibited a morphology similar to p-preDC. The majority of FC exhibited a characteristic plasmacytoid morphology, with a round shape, a smooth surface, and an eccentric nucleus on Wright-Giemsa staining (FIG. 2c). Transmission electronic microscopy confirmed the plasmacytoid morphology for the majority of FC (FIG. 2d). In the face of these striking phenotypic and morphological similarities, the present inventor hypothesized that the predominant CD11c^{dim}/B220⁺/CD11b⁻ cell population in the FC gate is likely the equivalent to the resting CD8α⁺p-preDC, a residual subpopulation of bone marrow p-preDC^{26, 32}.

### Example 3

### FC Behave Similarly to p-preDC after CpG ODN Stimulation

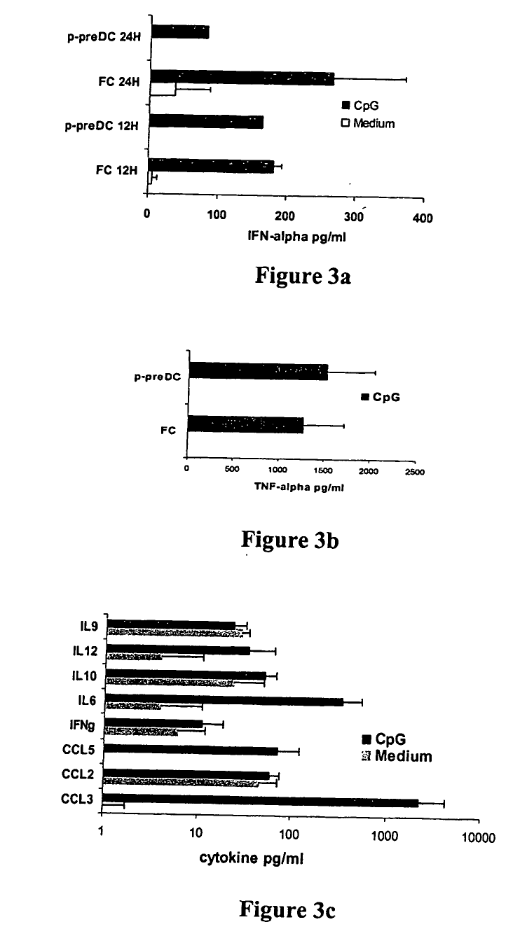

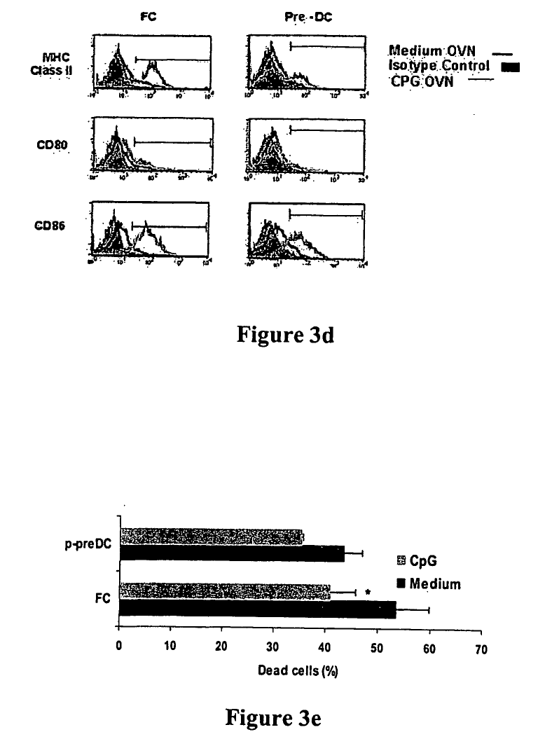

Given that IFN-α, TNFα and inflammatory cytokine production are main features of p-preDC, the present inventor examined whether FC resemble p-preDC in response to stimulation with CpG ODN. FC produced IFN-α after CpG ODN stimulation at levels similar to those produced by p-preDC (FIG. 3a). Additionally, as is the case for pre-DC, FC did not produce significant levels of IFN-α after LPS stimulation (data not shown). In addition to IFN-αsecretion, FC responded to CpG ODN stimulation by producing large amounts of TNF-α (FIG. 3b), and other pro-inflamatory cytokines including high amounts of Mip1-αCCL3, moderate amounts of IL-6 and RANTES/CCL5, and low levels of IL-12p70 (FIG. 3c). FC produced low amounts of IL9, IL-10, IFN-γ, and MCP-1/CCL2 (FIG. 3c) and no GM-CSF, IL-1β, IL-2, IL-4, IL-5 or IL-13 (data not shown) either after culture with medium or CpG stimulation. In total, these data demonstrate that FC respond to CpG ODN as is reported for p-preDC.

Because p-preDC mature after CpG ODN stimulation, so the inventor also analyzed whether FC resemble p-preDC in maturation/activation^{30, 32,33}. After an overnight exposure with medium or CpG ODN, FC were analyzed for MHC-class II, CD80 and CD86 expression (FIG. 3d). Class II and CD86 were highly upregulated on FC (from 18±8 to 74±6.5% and from up to 7±19 to 86±12%, respectively, n=4), and, to a lesser extent, CD80 (from 10±3.7 to 23.5±8%, n=4) (FIG. 3d). Similarly, CD86 is upregulated on p-preDC (from 12±9 to 85±13%, n=2) in a similar amount to FC, but Class II upregulation on p-preDC (from 10±2 to 39±4%, n=2) did not increase as much as it did on FC. The increase of CD80 (from 7±1% to 12.5±1%, n=2) on p-preDC is only slight.

In addition to phenotypic maturation, the inventor analyzed whether CpG stimulation could increase FC survival after overnight culture, as has been published for p-preDC²⁶. FC and p-preDC were cultured overnight with medium only or CpG ODN. Stimulation of FC with CpG decrease mortality by 10% compared to media alone (43.5±3.5%, and 53±6%, respectively, P=0.0038) (FIG. 3e). Similarly, mortality in p-preDC was significantly decreased after stimulation with CpG compared with media alone (35±0.3% versus 43.5±3.5% dead, P=0.024). Therefore, FC resemble p-preDC in their high sensitivity to death in culture and in their improved survival after CpG ODN stimulation. Collectively, these data show that FC share numerous functional characteristics with p-preDC that include nor only phenotype and morphology, but also in vitro function.

### Example 4

### FL is a Key Cytokine for FC Expansion and Maturation In Vitro

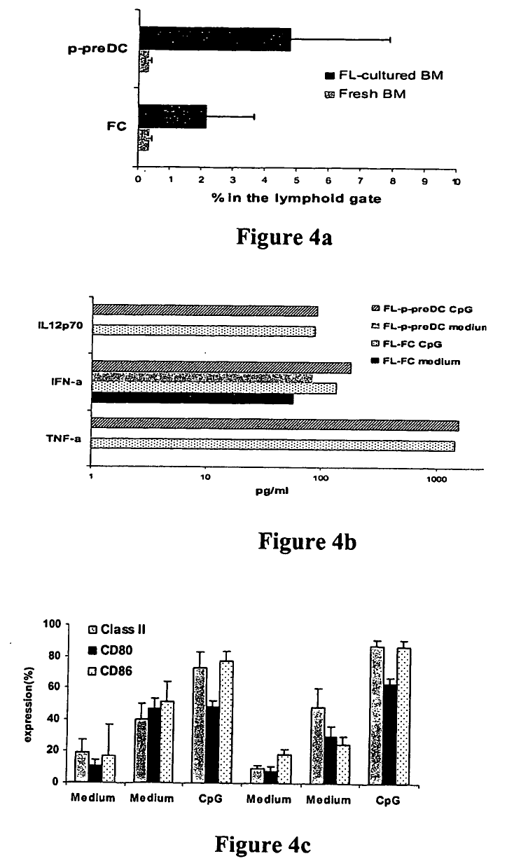

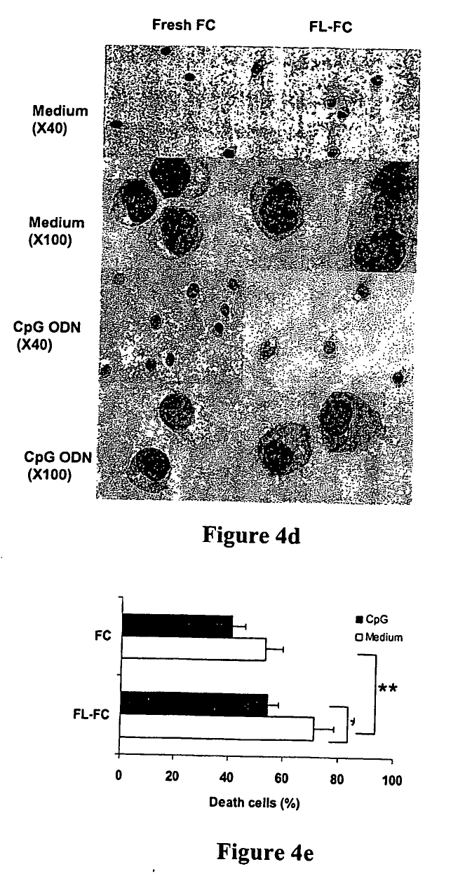

Co-culture of BM cells (BMC) with FL increases the frequency of p-preDC.^{30, 31}. The inventor then determined whether FC can be propagated using similar culture conditions. After 10 days in culture with FL, FC (FL-derived FC), and p-preDC (FL-derived p-preDC) were sorted. A 7-fold increase in the FL-derived FC (n=8) and 17-fold in FL-p-preDC (n=4) from the cultured BM (FIG. 4a).

Interestingly, FL-derived FC were in a more activated state than fresh FC, as evidenced by their morphology (FIG. 4b). Dendrites were already beginning to appear on FL-derived FC after overnight culture, and their appearance was amplified after exposure to CpG ODN. The effect of FL-treatment on FC maturation was also demonstrated by the ability of FL-derived FC to produce significant amounts of IFN-α after overnight culture (FIG. 4c). P-preDC derived from FL bone marrow culture (FL-derived p-preDC) also produced IFN-α after overnight culture. Stimulation with CpG-ODN overnight increased further the IFN-α secretion, as well as TNF-α, or IL-12p70, production.

The effect of FL on FC or p-preDC was further evaluated by analyzing at activation marker expression after overnight culture. Interestingly, both FL-derived FC and FL-derived p-preDC significantly upregulated their expression of MHC-class II, CD80, and CD86, as compared to FC and p-preDC sorted from fresh BMC (FIG. 4d). Indeed, 40% of FL-derived FC expressed Class II versus 18% of fresh FC, 47% expressed CD80 versus 11% of fresh FC, and 51% expressed CD86 versus 17% of fresh FC. Similarly, FL-derived p-preDC upregulated the level of Class II, CD80 and CD86. CpG ODN exposure overnight further increased the level of expression of these activation markers on both FL-derived FC (73% of Class II, 48% of CD80 and 77% expression of CD86) and FL-derived p-preDC (88% of Class II, 63% of CD80, and 87% of CD86) (FIG. 4d).

FL-derived FC are even more sensitive to death after overnight culture than fresh cells (71% versus 53.6% respectively, n=3, P=0.0032) (FIG. 4e). As for fresh FC and the exposure to CpG ODN overnight significantly decreased mortality of FL-treated FC by 10-15%, (P=0.02155). Interestingly, FL-derived p-preDC were also more sensitive to death after overnight culture than fresh cells, and were also partially rescued by CpG exposure (data not shown). In conclusion, FC as well as p-preDC expanded from FL-supplemented BM cell cultures are in a more advanced maturation/activation stage than freshly isolated cells. Nevertheless, they still display similar cytokine secretion, activation marker upregulation and survival patterns after CpG ODN exposure.

### Example 5

### In Vivo FL-Mobilized-FC (Containing More than 90% of CD11c⁺) Facilitate HSC Engraftment in Allogeneic Recipients

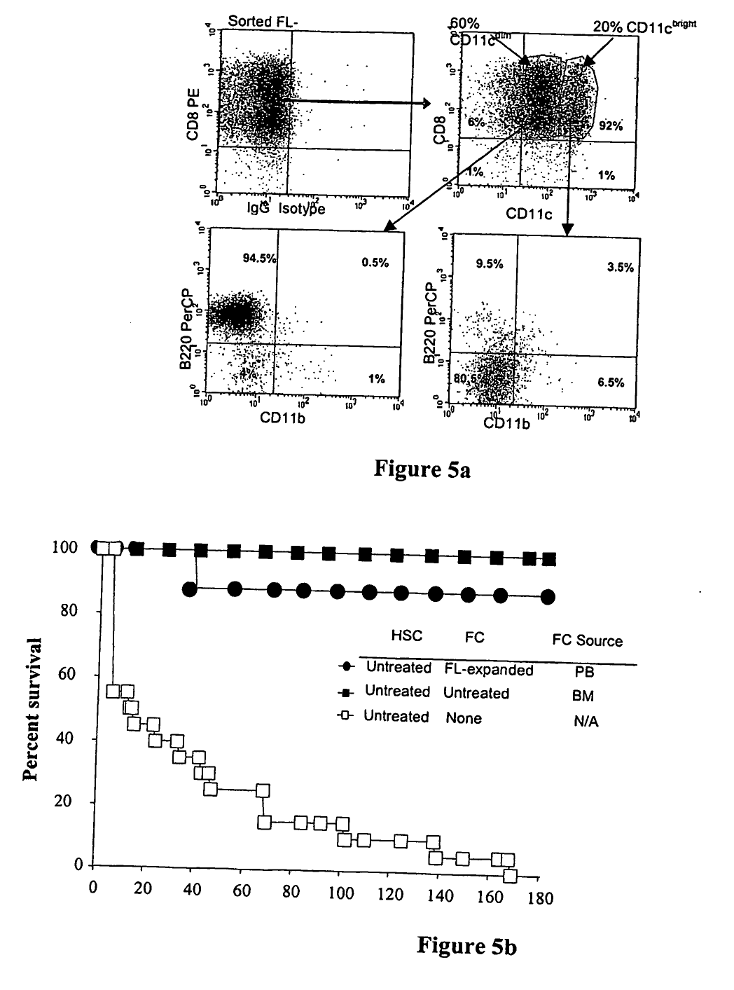

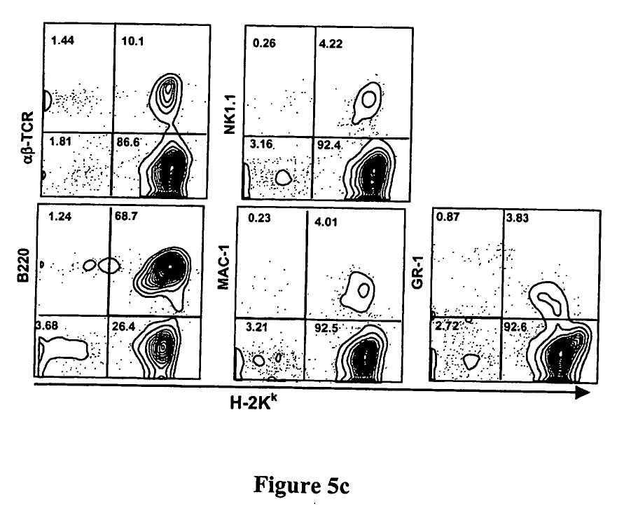

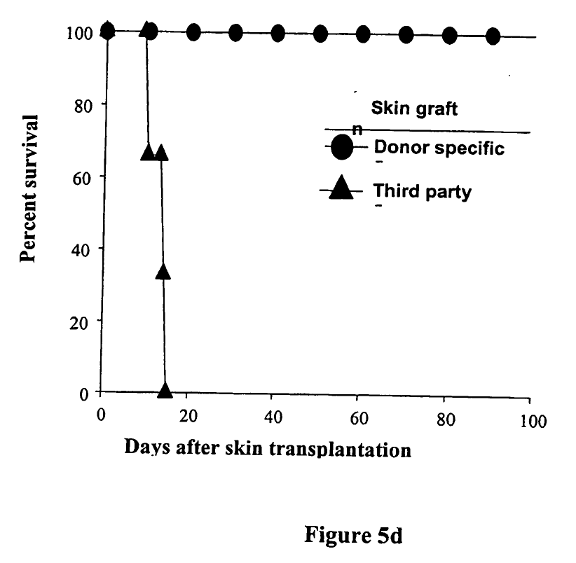

Several studies have shown that FL-treatment in vivo expands dendritic cells, including the p-preDC subtype^{27, 34}. It was previously shown that mice treated with FL demonstrate a significant expansion of FC in PB, BM and spleen, with the peak production at 10 days³⁵. The present invention characterizes the influence of FL administration on the different subtypes in the FC population in PB and analyzed the functional potential of purified blood FL-mobilized FC (FL-FC) to facilitate HSC engraftment. Approximately 85-90% of FL-FC in the PB express CD11c (FIG. 5a), and 5-7% express NK1.1, but none express CD19 or CD14 (data not shown). Interestingly, there were clearly two distinct DC populations: CD11c^{dim} and CD11c^{bright}. Further analysis showed that the 60% CD11c^{dim} population, characteristic of an immature DC phenotype, presented only the p-preDC phenotype (B220⁺/CD11c^{dim}/CD11b⁻). The 20% CD11c^{bright} population, characteristic of mature DC, contained a majority of mature lymphoid DC (B220/CD11c^{dim}/CD11b⁻) and all expressed the CD86 marker (data not shown). Therefore, FL mobilization induced a significant increase in the CD11c population, and dramatically decreased the B cell, and monocyte populations (data not shown).

Next, it was determined whether purified FL-FC from PB maintained their ability to facilitate HSC engraftment in allogeneic recipients. HSC were sorted from the marrow of untreated B10.BR mice and FC (CD8⁺/TCR⁻) from the PB of FL-treated B10.BR mice after 10 days of treatment. Allogeneic (C57BL/10, H-2^{b}) recipient mice were ablatively conditioned and reconstituted with 5,000 HSC plus 30,000 FL-FC. Control C57BL/10 mice received 5,000 HSC alone or 5,000 HSC plus 30,000 FC from untreated B10.BR donor mice. FL-FC were functional, as evidenced by 87% long-term survival (>180 days) (FIG. 5b). All mice receiving FC from untreated mice with HSC survived longer than 180 days. In contrast, none of the mice receiving allogeneic HSC alone survived after greater than or equal to 170 days. Thus, the FL-FC from PB, were functional in enabling the engraftment of HSC in allogeneic recipients.

As donor cell engraftment is considered to be an indicator of allograft tolerance, recipients of HSC plus FL-FC for donor chimerism and multiple hematopoietic lineages were examined 3 months after transplantation. All surviving animals tested showed >95% donor chimerism for multiple lineages, including T cells, NK cells, B cells, macrophages, and granulocytes (FIG. 5c).

To test whether these FL-FC plus HSC chimeras were functionally tolerant, skin grafts from B10.BR (HSC donor) or BALB/c (third party) mice were performed. Donor-specific skin grafts were accepted by the chimeras (MST≧100 days), while third-party (BALB/c) grafts were promptly rejected (MST=15 days) (FIG. 5d). In conclusion, FL-treatment significantly expands FC in PB that consist of 85-90% CD11c⁺ cells, with 20% of these being mature DC, and the distinct majority (60-65%) resembling the p-preDC phenotype. Most importantly, FL-FC enhance HSC engraftment and tolerance induction in allogeneic recipients.

### Example 6

### Purified p-preDC Facilitate HSC Engraftment in Allogeneic Recipients with Less Efficiency than FC

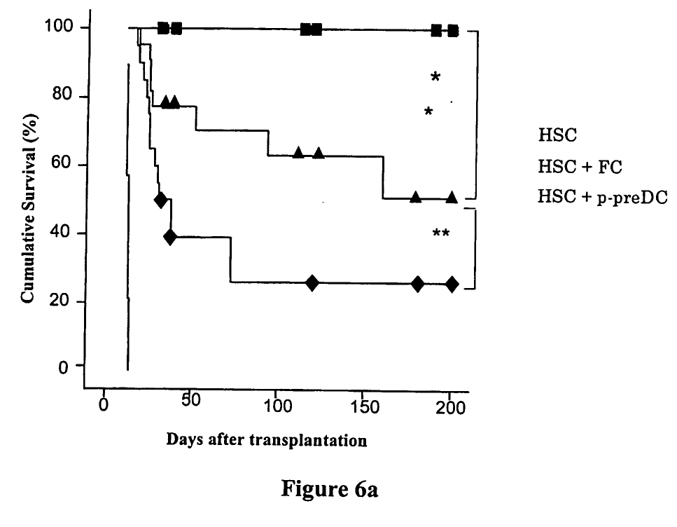

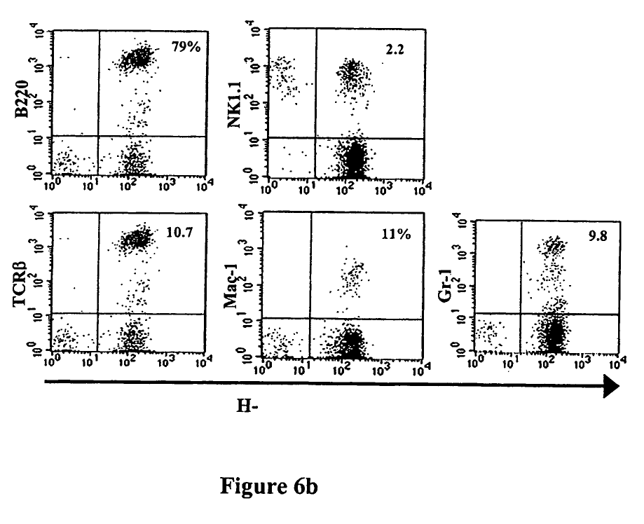

Due to the predominance of p-preDC in either fresh BM FC or FL-mobilized FC, it was determined whether purified p-preDC from BMC were able to facilitate HSC engraftment. 5,000 C57BL/6J (H2K^{b}) HSC were injected alone into ablated C3H (H2K^{k}) mice (HSC group), or with 30,000 C57BL/6J F (HSC+FC group), or with 30,000 C57BL/6J p-preDC (HSC+p-preDC group). The 200 day survival was 20% for the HSC alone group (n=20) with a median survival of 30 days (FIG. 6a). The engraftment was significantly enhanced in recipients of HSC+p-preDC (51% survival, n=22) compared to the HSC alone group (P=0.0076). However, the survival was significantly enhanced in the HSC+FC group (95% survival, n=21) compared to the HSC alone group (P<0.00001) and was also significantly enhanced over the HSC+p-preDC group (P=0.0083). Thus, p-preDC effectively facilitated HSC engraftment, but less efficiently than the FC total. Interestingly, donor-derived hematopoetic cells were detectable at levels >95% in all of the live animals receiving HSC+p-preDC at 3 months after transplantation (FIG. 6b), indicating that the chimerism was significantly high and consistent between all the recipients. Multilineage production for myelo/monocytes and T/B/NK cells was present in animals transplanted with HSC+p-preDC at three months transplantation (FIG. 6b).

Survival of skin grafts in HSC+p-pre-DC chimeras (C57BL/6J→C3H/HeJ). To test whether the chimerism achieved with transplantation of HSC+p-preDC induced donor-specific tolerance, skin grafts from C57BL/6J (HSC donor) or BALB/c (third party) mice were performed. Donor-specific (C57BL/6J, n=8) and third-party (BALB/c, n=8) skin grafts were performed 3 months after HSC plus p-pre-DC transplantation. Data was collected for skin graft survival over 50 days. Donor-specific skin grafts were accepted by chimeras (MST)≧50 days), while third-party (BALB/c) grafts were promptly rejected (MST=13.5 days). In conclusion, p-preDC enhance engraftment of purified allogeneic HSC, as shown by chimerism and tolerance to donor antigens, but with somewhat less efficiency than FC over time.

Materials and Methods

Mice C57BL/6 (B6;H2^{b}), B10.BR (H2^{k}), BALB/c (H2^{d}), and gene knockout (KO) and transgenic strains were purchased from Jackson Laboratories (Bar Harbor, Me.) and Taconic Laboratories (Germantown, N.Y.) or generated through in-house breeding. These B6 congenic knockout strains include αβ-TCR; γδ-TCR; CD3ε; and CD3δ. The CD3ε transgenic was derived from insertion of the human CD3ε transgenic in a B6 mouse (B6 CBA-Tgn). Animals were housed in a barrier animal facility at the Institute for Cellular Therapeutics, University of Louisville, and cared for according to specific National Institutes of Health animal care guidelines.

HSC and FC isolation and transplantation. Bone marrow isolated from tibias and femurs was resuspended at a concentration of approximately 100×10⁶ cells/mL in sterile Cell Sort Media (CSM): Hank's Balanced Salt Solution without phenol red (Gibco), 2% heat inactivated fetal calf serum (Gibco), 2 μL/mL HEPES buffer (Gibco) and 30 μL/mL of Gentimicin (Gibco). Directly labeled monoclonal antibodies (Pharmingen) were added at appropriate saturating concentrations, and the sample was then incubated at 4° C. for 30-45 minutes, washed, filtered and resuspended to 2.5×10⁶ cells/mL. For HSC, antibodies included Sca-1 (Ly6A/E)-PE, c-Kit (CD117)-APC, and the FITC-conjugated anti-lineage antibodies: B220 (CD45R), CD8α (53-6.7), MAC-1 (CD11b) (M1/70), GR-1 and β-TCR(H57-597). For GFP+ HSC sorting, the antibodies for Lineage markers above were conjugated to APC, while those to c-Kit were APC-Cy7. For FC, antibodies included, anti-CD8α (53-6.7)-PE, anti-βTCR (H57-597)-FITC and anti-γδTCR (GL3)-FITC antibodies. FC analyses were preblocked with CD16 (24G2)-unlabeled. Stained cells were sorted by multi-parameter live sterile sorting on a FACS-Vantage flow cytometer (Becton Dickinson). For HSC, Sca-1⁺/c-Kit⁺/Lin⁻ cells were collected from within the conventional lymphoid gate. The FC (CD8⁺/TCR⁻ cells) and T cells (CD8⁺/TCR⁺ cells) were collected within the conventional lymphoid gate. Cells were analyzed post sorting, and only samples of greater than 95% purity were centrifuged and resuspended in MEM, then transplanted.

Transplantation. Allogeneic recipient mice were conditioned with 950 cGy of TBI from a Cesium source (Nordion, Ontario, Canada) and reconstituted with 10,000 HSC+/−30,000 CD8⁺/TCR⁻ or 30,000 CD8⁺/TCR⁺ cells by tail vein injection. Chimerism was detected by flow cytometric analysis at 2, 4 and 6 months using antibodies H-2 K^{b}-PE (AF6-88.5), and anti-H-2K^{k} (AF3-12.1). Syngeneic mice received 1000 HSC, or 500 HSC plus 30,000 FC or CD8⁺/TCR⁻ T cells.

Significance estimates. Graft survival was calculated according to the Kaplan-Meier method, and statistical significance was determined by application of the Student's T test. All graphs of transplanted mice represent experimental animals from at least two (and in most cases, three) separate days of sorting/transplantation.

Skin graft. Skin grafting was performed by a modification of the method of Billingham [58]. Full-thickness tail skin grafts were harvested from the tails of B10.BR (H2^{k}, donor-specific) and BALB/c (H2^{d}, third-party) mice. Recipient mice were anesthetized with Nembutal (pentobarbital sodium injection; Abbott, North Chicago, Ill.), and full-thickness graft beds were prepared surgically in the lateral thoracic wall, preserving the panniculus carnosum. The grafts were covered with a double layer of Vaseline gauze (Alba-Waldensian, Rockwood, Tenn.) and a plaster cast. Casts were removed on the seventh day; and grafts were scored by daily inspection for the first month and then weekly thereafter for the percentage of rejection, as reflected by petechial and eschar formation. At the time of cast removal, grafts were inspected for vascular perfusion, absence of infection, and technical success. Rejection was defined as complete when no residual viable graft could be detected.

Reverse transcription PCR and southern blot analysis. Single cell suspensions from the bone marrow of three mice was combined and stained for FC (as above). Double sorted cells (2.5×10⁴, 99% of purity) were washed in PBS, and dry pellets were frozen in nitrogen liquid. Total RNA was extracted according to the manufacture's protocol (Rneasy Mini Kit; QIAGEN, Valencia, Calif.). During RNA purification, on-column DNase digestion with the RNase-free DNase SET (Qiagen) was done to remove contaminating DNA. First strand cDNA was prepared with Oligo (dT) primer and reverse transcribed with a SUPERSCRIPT first-strand synthesis system for RT-PCR (Invitrogen Corporation, Carlsbad, Calif.). The PCR reaction volume was 50 μL, containing 5 μL of cDNA, 0.4 μM of each primer, 3 μL of 25 mM MgCl2, 1 μL of 10 mM mixed dNTP, 2 U of Taq DNA Polymerase (Promega Corporation, Madison, Wis.). Primers are listed in Table 1.

TABLE 1

Sequence of oligonucleotides
used in reverse-transcriptase-coupled
polymerase chain reaction.

SEQ.
ID

| | | | | |
|---|---|---|---|---|
| GENE | STRAND | NO. | PRIMER SEQUENCE | PRODUCT |

| | | | | |
|---|---|---|---|---|
| β-actin | forward | 2 | 5′-TGTGATGGTGGGAATGGG | 514 bp |
| | | | TCAG-3′ | |
| | reverse | 3 | 5′-TTTGATGTCACGCACGAT | |
| | | | TTCC-3′ | |
| | probe | 4 | 5′-TGTTACCAACTGGGACGA | |
| | | | CA-3′ | |
| CD3δ | forward | 5 | 5′-CTCCTGGCTTTGGGCGTC | 203 bp |
| | | | TACTG-3′ | |
| | reverse | 6 | 5′-TTGCTATGGCACTTTGAG | |
| | | | AAACCTCC-3′ | |
| | probe | 7 | 5′-GCCTCTTCGAGATCGTGA | |
| | | | AG-3′ | |
| CD3ε | forward | 8 | 5′-ACCTGACAGCAGTAGCCA | 192 bp |
| | | | TAATCATC-3′ | |
| | reverse | 9 | 5′-GCTCATAGTCTGGGTTGG | |
| | | | GAACAG-3′ | |
| | probe | 10 | 5′-ATCACTCTGGGCTTGCTG | |
| | | | AT-3′ | |
| TCRα | forward | 11 | 5′-GCTCTCCTTGCACATCAC | 502 bp |
| | | | AG-3′ | |
| | reverse | 12 | 5′-AAATCCGGCTACTTTCAG | |
| | | | CA-3′ | |
| | probe | 13 | 5′-GGAGCAACCAGACAAGCT | |
| | | | TC-3′ | |
| TCRβ | forward | 14 | 5′-ATGAGCTGCAGGCTTCTC | 487 bp |
| | | | CT-3′ | |
| | reverse | 15 | 5′-CGAGGGTAGCCTTTTGTT | |
| | | | TG-3′ | |
| | probe | 16 | 5′-CCCAGACAGCTCCAAGCT | |
| | | | AC-3′ | |

cDNA was amplified with a Gene Amp PCR System 2400 (PerkinElmer Life Science, Gaithersburg, Md.; Applied Biosystems, Foster City, Calif.) by an initial denaturation step of 94° C. for 3 minutes; followed by 40 cycles of 94° C. for 1 minutes, 60° C. for 2 minutes, 72° C. for 3 minutes, and a final elongation step of 72° C. for 10 minutes. Half of each PCR reaction was separated throughout a 2% agarose electrophoresis gel containing ethidium bromide and blotted onto nylon transfer membrane (Fisher Scientific, Montreal, Canada). Prehybridization of membrane was conducted for 2 hours at 45° C. in pre-hybridization buffer (5×SSC, 0.02% SDS, 1% Blocking Buffer from Roche Molecular Biochemicals (Indianapolis, Ind.). cDNA probes were labeled with Digoxigenin-ddUTP kit (Roche Molecular Biochemicals) according to the manufacture's protocol. DIG-probes were added directly to membrane in pre-hybridization buffer overnight at 45° C. Membranes were stringently washed 2 times in 6×SSC for 30 minutes at 65° C. Luminescence was detected with CSPD (DIG luminescent detection kit for nucleic acids, Roche Molecular Biochemicals) after exposure on BIOMax MS film (Fisher Scientific). These analyses were repeated three times with similar results.
Results

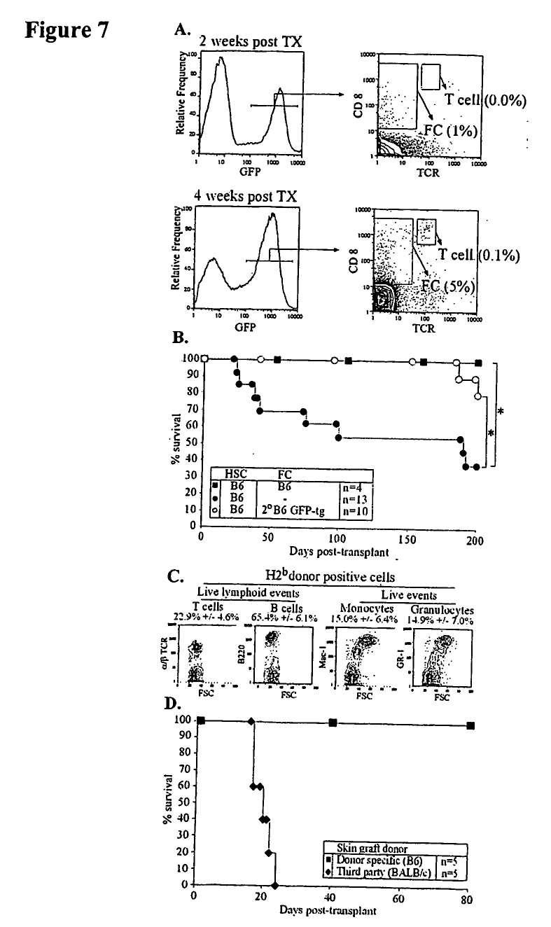

CD8⁺/TCR⁻ FC are HSC derived. To establish that FC are derived from HSC, c-Kit⁺/Sca-1⁺/lin⁻ HSC from GFP⁺ donors (H-2^{b}) [59] were purified and transplanted 10,000 HSC into syngeneic recipients conditioned with 950 cGy TBI. At two and four weeks, GFP⁺ FC were enumerated. Notably, GFP⁺ cells contained FC, confirming a bone-marrow-derived origin (FIG. 7A). To demonstrate function, GFP⁺ FC from older animals were sorted from the marrow and co-administered with 10,000 HSC to conditioned allogeneic secondary recipients. As expected, these GFP⁺ FC were functional to facilitate HSC engraftment in allogeneic recipients with donor chimerism of 99% by 2 months post transplant (FIG. 7B). Moreover, these mice displayed durable multilineage blood cell production (FIG. 7C). Notably, recipients of purified HSC plus FC were tolerant to donor-specific skin allografts (FIG. 7D).

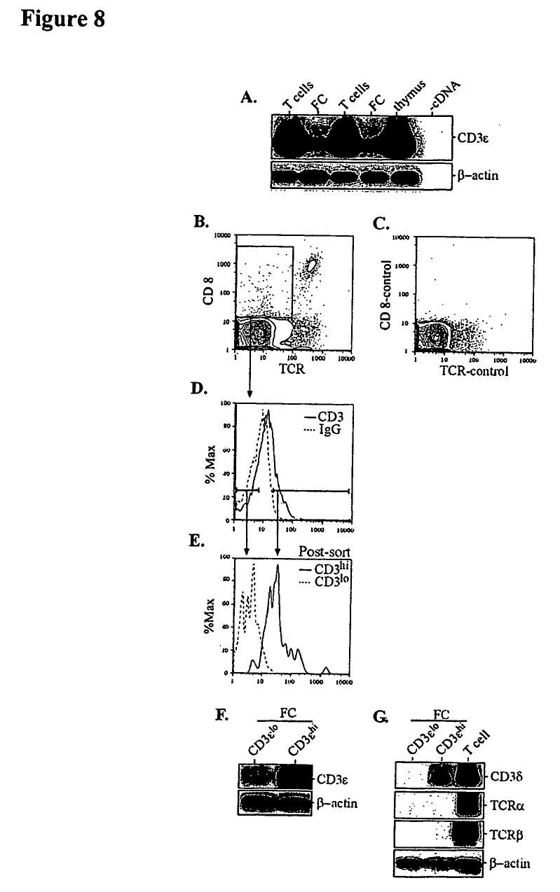

CD8⁺/TCR⁻ FC express CD3ε. It was previously reported that CD3ε⁺ FC facilitate engraftment of HSC in allogeneic recipients. Approximately 5% of the total cells within the FC gate express CD3ε [9]. The level of CD3ε expression is dimmer than for conventional T cells, suggesting a population separate from T lymphocytes [9, 48]. To further evaluate the role of CD3ε in FC function, FC for expression of CD3ε gene transcripts was examined herein using RT-PCR. FC were double sorted to >99% purity from the combined bone marrow of three mice. CD8⁺/TCR⁺ bone marrow cells and thymocytes were used as controls. RT-PCR analysis was performed for β-actin. The products of the reaction were Southern blotted and probed with a target-specific oligonucleotide probe, quantified, and the cDNA were normalized to the signal (FIG. 8A). As expected, abundant RT-PCR products for CD3ε were detected in control T cell and thymocyte cDNA. Similarly, the bulk population of CD8⁺/TCR⁻ FC contained readily detectable CD3ε transcript (FIG. 8A).

Next, the analyses was redefined to attempt to detect the FC receptor complex components within FC that express more or less CD3ε by flow cytometry. CD3ε^{hi} FC and CD3ε^{lo} FC were sorted (FIG. 8B-E). The inventor discovered that the transcript for CD3ε is expressed in both CD3ε^{hi} FC and CD3ε^{lo} FC (FIG. 8F). However, because flow cytometric separation of a continuous gradient of expression is not perfect, the possibility that signal in the CD3ε^{lo} FC is due to contamination by CD3ε^{hi} FC cannot be excluded. Nevertheless, neither TCRα nor TCRβ products were visible in probed RT-PCR analyses of either of the FC cDNA samples, even when the films were purposely overexposed (FIG. 8G), confirming that the samples were not contaminated with T cells and further suggesting a separate, non T cell ontogeny for FC. As expected, TCRα nor TCRβ transcripts were detected in control T cell samples (FIG. 8G). Interestingly, only CD3ε^{hi} FC contained the transcript for CD3δ. These data therefore suggest that CD3ε^{hi} FC express a functional CD3 complex and that CD3ε^{hi} FC may be a more mature or activated developmental state compared to the CD3ε^{lo} FC population in light of the presence of CD3δ in that population. Alternatively, CD3ε^{hi} FC could be a separate population.

The CD3ε complex is critical to development of functional FC for allogeneic transplantation. To further define the role of CD3ε in allogeneic facilitation, mice defective in production of various CD3 complex components were utilized: CD3ε transgenic (CD3ε-tg); CD3ε KO (CD3ε^{Δ-/Δ-}) and CD3δ KO (CD3δ^{-/-}). The resulting block in production of T and NK cells for each of the mutants is illustrated in Table 2.

TABLE 2
Lymphoid cells produced in genetically altered mice.

| | Lymphoid cells produced | | |
|---|---|---|---|
| Mouse Strain | λδ T cells | αβ T cells | NK |
| B6 CD3ε-tg | − | − | − |
| B6 CD3ε^{Δ-/Δ-} | − | − | + |
| B6 CD3δ^{-/-} | + | −/+ | + |
| B6 TCRβ^{-/-} | + | − | + |
| B6 TCRα^{-/-} | + | − | + |

The presence (+), absence (−), or severe reduction (−/+) of lymphoid populations (γδ T cells, αβ T cells, and NK cells) in genetically manipulated strains of mice is indicated. The expression of both CD3ε and CD3δ are necessary for development of a functional CD3 complex [60, 61]. Introduction of human CD3ε into transgenic mice (CD3ε-tg) leads to CD3ε-promoter-driven overexpression of human CD3ε, resulting in impaired formation of an active CD3 signaling complex and a profound block in the development of T cells and NK cells [62].

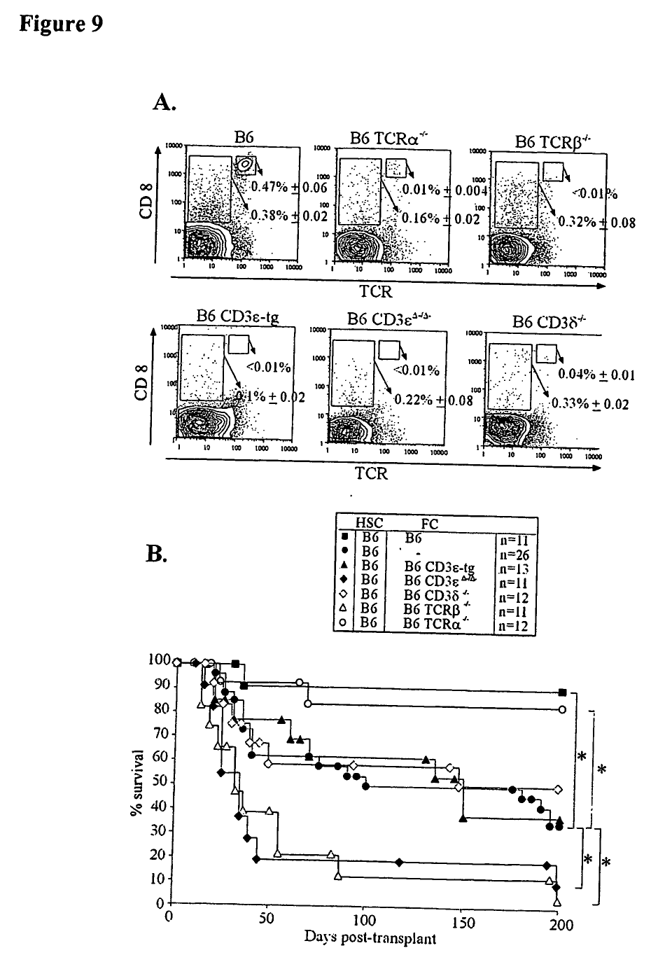

Mice with mutations in CD3 complex genes have CD8⁺/TCR⁻ FC (FIG. 9A). To determine if the FC from CD3ε-tg mice are functional, 30,000 FC were transplanted with 10,000 wild type HSC into ablated B10.BR allogeneic recipients. Notably, CD3ε-tg FC do not facilitate, as evidenced by a pattern of engraftment equivalent to HSC alone (P=0.86, FIG. 9B). In contrast, control CD8⁺/TCR⁻ FC from age and sex matched wild type B6 mice significantly facilitate the engraftment of HSC compared to HSC alone (P=0.0061, FIG. 9B). It is possible that CD3ε-expressing cells are required to engender proper FC maturation, or alternatively that a functional CD3 complex on FC is required to facilitate HSC engraftment.

To confirm that the dysfunction of cells with an FC phenotype in the CD3ε-tg mice was due to the disruption of the CD3ε complex, CD3 loss-of-function mutants were also examined, each of which has a unique developmental block. CD3ε^{Δ-/Δ-} mice, which lack T cells due to the specific deletion of the CD3ε gene [53], also do not produce functional FC (FIG. 9B). In fact, co-administration of FC from CD3ε^{Δ-/Δ-} mice with wild type HSC significantly impairs survival compared to HSC alone (P=0.027).

While CD3ε is a critical component of both the γδ and αβ T cell receptors, CD3δ transmits a mitogen-activated protein-kinase signal that is required only for αβ T cell development [61]. CD3δ^{-/-} mice have impaired production of αβ-TCR⁺ T cells, but no defect in production of γδ T cells [61]. Notably, CD8⁺/TCR⁻ FC from CD3δ^{-/-} mice also do not facilitate in comparison to HSC alone (P=0.86, FIG. 9B). Survival was significantly reduced compared to that for wild type control FC plus HSC (P<0.05). However, in contrast with the CD3ε mutants, survival was not significantly impaired compared to HSC alone.

Production of functional FC is independent of αβ-TCR⁺ T cells. Two hypotheses could explain the requirement for the CD3ε complex in FC function. It is possible that genetic defects in the CD3 complex may generate an intrinsic defect in CD8⁺/TCR⁻ FC through impaired development of the CD3ε/FCp33 receptor complex [48]. Alternatively, development of functional FC may require the presence of αβ-TCR⁺ T cells for activation and/or maturation of FC to occur. To address these possibilities, FC from mice mutant for TCRα or TCRβ were examined, both of which lack αβ T cells. Both strains contained CD8⁺/TCR⁻ FC (FIG. 9A). As previously reported, FC from TCRβ^{-/-} mice did not facilitate survival (FIG. 9B). In fact, TCRβ^{-/-} FC significantly impaired survival compared to HSC alone (P=0.004).

To eliminate the possibility that αβ T cells are required to elicit maturation and effector function of FC, FC from mice deleted for the TCRα gene were examined. TCRα^{-/-} mice selectively do not produce αβ T cells (Table 2). Strikingly, FC from TCRα^{-/-} mice increased survival of B10.BR recipients in comparison to HSC alone (P=0.023, FIG. 9B). Thus, a deficiency in αβ T cells is not a viable explanation for the impaired FC function observed in the CD3-complex mutants, pointing to a cell autonomous (intrinsic) or developmental defect for FC obtained from CD3ε mutant donors.

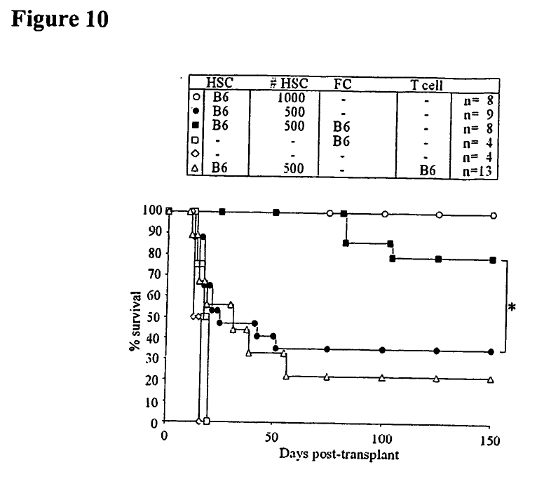

CD8⁺/TCR⁻ FC facilitate engraftment of suboptimal numbers of HSC in syngeneic recipients. To evaluate the mechanism of FC function in the absence of alloreactivity, the inventor evaluated whether FC enhance engraftment of HSC in syngeneic recipients. The minimum number of HSC for engraftment were first established. Transplantation of less than 1,000 HSC results in significantly reduced long-term survival of syngeneic B6 recipients conditioned with 950 cGy TBI (FIG. 10). Strikingly, the addition of 30,000 B6 FC to the 500 HSC significantly increased survival (P=0.0034, FIG. 10). Neither irradiated control mice nor those transplanted with FC alone survived (FIG. 10). Importantly, conventional T cells could not substitute for FC (FIG. 10). Thus, FC but not T cells potently facilitate engraftment of suboptimal numbers of HSC in the absence of alloreactivity.

Taken together, these data clearly distinguish FC from T cells. Moreover, they indicate that FC require the CD3ε gene to facilitate allogeneic HSC engraftment. The unique function(s) of FC make them an attractive focus for new cell-based therapeutic approaches to enhance HSC engraftment while reducing toxicity, especially when limiting numbers of HSC are available.

The foregoing description is considered as illustrative only of the principles of the invention. Further, since numerous modifications and changes will be readily apparent to those skilled in the art, it is not desired to limit the invention to the exact construction and process shown as described above. Accordingly, all suitable modifications and equivalents may be resorted to falling within the scope of the invention as defined by the claims that follow.

The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

### REFERENCE LIST

1. Ildstad, S. T. & Sachs, D. H. Reconstitution with syngeneic plus allogeneic or xenogeneic bone marrow leads to specific acceptance of allografts or xenografts. Nature 307, 168-170 (1984).
2. Billingham, R. E., Brent, L., & Medawar, P. B. Actively acquired tolerance to foreign cells. Nature 172, 603-607 (1953).
3. Lu, L., Shen, R. N., & Broxmeyer, H. E. Stem cells from bone marrow, umbilical cord blood and peripheral blood for clinical application: current status and future application. Crit Rev. Oncol. Hematol. 22, 61-78 (1996).
4. Korbling, M. & Anderlini, P. Peripheral blood stem cell versus bone marrow allotransplantation: does the source of hematopoietic stem cells matter? Blood 98, 2900-2908 (2001).
5. Champlin, R. T-cell depletion to prevent graft-versus-host disease after bone marrow transplantation. Hematology/Oncology Clinics of North America 4, 687-698 (1990).
6. Blazar, B. R. et al. T-cell depletion of donor marrow grafts: Effects on graft-versus-host disease and engraftment New York, N.Y. Liss, 1987).
7. Drobyski, W. et al. Effect of T-Cell Depletion as Graft-versus-Host Disease Prophylaxis on Engraftment, Relapse, and Disease-Free Survival in Unrelated Marrow Transplantation for Chronic Myelogenous Leukemia. Blood 83, 1980-1987 (1994).
8. Gandy, K. L., Domen, J., Aguila, H. L., & Weissman, I. L. CD8⁺TCR⁺ and CD8⁺TCR⁻ cells in whole bone marrow facilitate the engraftment of hematopoietic stem cells across allogeneic barriers. Immunity 11, 579-590 (1999).
9. Kaufman, C. L. et al. Phenotypic characterization of a novel bone-marrow derived cell that facilitates engraftment of allogeneic bone marrow stem cells. Blood 84, 2436-2446 (1994).
10. Colson, Y. L. & Wright, R. D. Facilitating cells: FCp33/TCR□ heterodimer expression and the absence of GVHD effector activity. Journal of the American College of Surgeons 195[3], S1-S115. 2002. Ref Type: Abstract
11. Grimes, H. L. et al. Graft facilitating cells: evidence for a distinct non-T cell lymphoid lineage. Experimental Hematology In Press (2004).
12. Morelli, A. E., Hackstein, H., & Thomson, A. W. Potential of tolerogenic dendritic cells for transplantation. Semin. Immunol 13, 323-335 (2001).
13. Thomson, A. W. & Lu, L. Dendritic cells as regulators of immune reactivity: implications for transplantation. Transplantation 68, 1-8 (1999).
14. Guermonprez, P., Valladeau, J., Zitvogel, L., Thery, C., & Amigorena, S. Antigen presentation and T cell stimulation by dendritic cells. Annu. Rev. Immunol 20:621-67., 621-667 (2002).
15. Banchereau, J. et al. Immunobiology of dendritic cells. Annu. Rev. Immunol. 18, 767-811. 2000.
16. Liu, Y. J. Dendritic cell subsets and lineages, and their functions in innate and adaptive immunity. Cell 106, 259-262 (2001).
17. Shortman, K. & Heath, W. R. Immunity or tolerance? That is the question for dendritic cells. Nat. Immunol 2, 988-989 (2001).
18. Kalinski, P., Hilkens, C. M., Wierenga, E. A., & Kapsenberg, M. L. T-cell priming by type-1 and type-2 polarized dendritic cells: the concept of a third signal. Immunol Today 20, 561-567 (1999).
19. Boonstra, A. et al. Flexibility of mouse classical and plasmacytoid-derived dendritic cells in directing T helper type 1 and 2 cell development: dependency on antigen dose and differential toll-like receptor ligation. J Exp Med 197, 101-109 (2003).
20. Coates, P. T. & Thomson, A. W. Dendritic cells, tolerance induction and transplant outcome. Am. J. Transplant 2, 299-307 (2002).
21. O'Connell, P. J. et al. Immature and mature CD8alpha+ dendritic cells prolong the survival of vascularized heart allografts. J Immunol 168, 143-154 (2002).
22. Arpinati, M., Green, C. L., Heimfeld, S., Heuser, J. E., & Anasetti, C. Granulocyte-colony stimulating factor mobilizes T helper 2-inducing dendritic cells. Blood 95, 2484-2490 (2000).
23. Rossi, M., Arpinati, M., Rondelli, D., & Anasetti, C. Plasmacytoid dendritic cells: do they have a role in immune responses after hematopoietic cell transplantation? Hum. Immunol 63, 1194-1200 (2002).
24 Liu, Y. J. & Blom, B. Introduction: TH2-inducing DC2 for immunotherapy. Blood 95, 2482-2483 (2000).
25. Siegal, F. P. et al. The nature of the principal type 1 interferon-producing cells in human blood. Science 284, 1835-1837 (1999).
26. Asselin-Paturel, C. et al. Mouse type I IFN-producing cells are immature APCs with plasmacytoid morphology. Nat. Immunol 2, 1144-1150 (2001).
27. Bjorck, P. Isolation and characterization of plasmacytoid dendritic cells from Flt3 ligand and granulocyte-macrophage colony-stimulating factor-treated mice. Blood 98, 3520-3526 (2001).
28. Fonteneau, J. F. et al. Activation of influenza virus-specific CD4+ and CD8+ T cells: a new role for plasmacytoid dendritic cells in adaptive immunity. Blood., (2003).
29. Kadowaki, N., Antonenko, S., Lau, J. Y., & Liu, Y. J. Natural interferon alpha/beta-producing cells link innate and adaptive immunity. J Exp Med 192, 219-226 (2000).
30. Gilliet, M. et al. The development of murine plasmacytoid dendritic cell precursors is differentially regulated by FLT3-ligand and granulocyte/macrophage colony-stimulating factor. J Exp Med 195, 953-958 (2002).
31. Brawand, P. et al. Murine plasmacytoid pre-dendritic cells generated from Flt3 ligand-supplemented bone marrow cultures are immature APCs. J Immunol 169, 6711-6719 (2002).
32. O'Keeffe, M. et al. Mouse plasmacytoid cells: long-lived cells, heterogeneous in surface phenotype and function, that differentiate into CD8(+) dendritic cells only after microbial stimulus. J Exp Med 196, 1307-1319 (2002).
33. Martin, P. et al. Characterization of a new subpopulation of mouse CD8alpha(+) B220(+) dendritic cells endowed with type 1 interferon production capacity and tolerogenic potential. Blood 100, 383-390 (2002).
34. Maraskovsky, E. et al. Dramatic increase in the numbers of functionally mature dendritic cells in Flt3 ligand-treated mice: multiple dendritic cell subpopulations identified. J Exp Med 184, 1953-1962 (1996).
35. Neipp, M., Zorina, T., Domenick, M. A., Exner, B. G., & Ildstad, S. T. Effect of FLT3 ligand and granulocyte colony-stimulating factor on expansion and mobilization of facilitating cells and hematopoietic stem cells in mice: kinetics and repopulating potential. Blood 92, 3177-3188 (1998).
36. Bilsborough, J., George, T. C., Norment, A., & Viney, J. L. Mucosal CD8alpha+ DC, with a plasmacytoid phenotype, induce differentiation and support function of T cells with regulatory properties. Immunology 108, 481-492 (2003).
37. Nikolic, T., Dingjan, G. M., Leenen, P. J., & Hendriks, R. W. A subfraction of B220(+) cells in murine bone marrow and spleen does not belong to the B cell lineage but has dendritic cell characteristics. Eur. J. Immunol. 32, 686-692 (2002).
38. Hochrein, H., O'Keeffe, M., & Wagner, H. Human and mouse plasmacytoid dendritic cells. Hum. Immunol. 63, 1103-1110 (2002).
39. Penna, G., Vulcano, M., Sozzani, S., & Adorini, L. Differential migration behavior and chemokine production by myeloid and plasmacytoid dendritic cells. Hum. Immunol 63, 1164-1171 (2002).
40. Kuwana, M., Kaburaki, J., Wright, T. M., Kawakarni, Y., & Ikeda, Y. Induction of antigen-specific human CD4(+) T cell anergy by peripheral blood DC2 precursors. Eur J Immunol 31, 2547-2557 (2001).
41. Rissoan, M. C. et al. Reciprocal control of T helper cell and dendritic cell differentiation. Science 283, 1183-1186 (1999).
42. Kuwana, M. Induction of anergic and regulatory T cells by plasmacytoid dendritic cells and other dendritic cell subsets. Hum. Immunol 63, 1156-1163 (2002).
43. Moore, K. W., de Waal, M. R., Coffman, R. L., & O'Garra, A. Interleukin-10 and the interleukin-10 receptor. Annu. Rev. Immunol. 19, 683-765 (2001).
44. Wakkach, A. et al. Characterization of dendritic cells that induce tolerance and T regulatory 1 cell differentiation in vivo. Immunity. 18, 605-617 (2003).
45. Wakkach, A., Cottrez, F., & Groux, H. Differentiation of regulatory T cells 1 is induced by CD2 costimulation. J. Immunol. 167, 3107-3113 (2001).
46. Kadowaki, N. et al. Distinct cytokine profiles of neonatal natural killer T cells after expansion with subsets of dendritic cells. J. Exp. Med. 193, 1221-1226 (2001).
47. Colson, Y. L. & Ildstad, S. T. Characterization of two unique cell surface molecules on the facilitating cell. Surg Forum 48, 481-484 (1997).
48. Schuchert, M. J., Wright, R. D., & Colson, Y. L. Characterization of a newly discovered T-cell receptor beta-chain heterodimer expressed on a CD8+ bone marrow subpopulation that promotes allogeneic stem cell engraftment. Nature Medicine 6, 904-909 (2000).
49. Huang, Y. et al. Matching at the MHC Class I K locus is essential for long-term engraftment of purified hematopoietic stem cells: a role for host NK cells in regulating HSC engraftment. Blood In Press (2004).
50. Billingham, M. E. Pathology and etiology of chronic rejection of the heart. Clin Transplantation 8, 289-292 (1994).
51. Storb R, Epstein R B, Bryant J, Ragde H, Thomas E D., Marrow grafts by combined marrow and leukocyte infusions in unrelated dogs selected by histocompatibility typing. Transplantation. 1968; 6:587-593.
52. Deeg H J, Storb R, Weiden P L, et al., Abrogation of resistance to and enhancement of DLA-nonidentical unrelated marrow grafts in lethally irradiated dogs by thoracic duct lymphocytes. Blood. 1979; 53:552-557.
53. Lapidot T, Faktorowich T, Lubin I, Reisnet Y., Enhancement of T-cell-depleted bone marrow allografts in the absence of graft-versus-host disease is mediated by CD8⁺ CD4⁻ and not by CD8⁻ CD4⁺ thymocytes. Blood. 1992; 80: 2406-2411.
54. Murphy W J, Bennett M, Kumar V, Longo D., Donor-type activated natural killer cells promote marrow engraftment and B cell development during allogeneic bone marrow transplantation. J Immunol. 1992; 148:2953-2960.
55. Martin P J., Donor CD8 cells prevent allogeneic marrow graft rejection in mice: Potential implications for marrow transplantation in humans. J Exp Med. 1993; 178:703-712.
56. Palathumpat V, Dejbakhsh-Jones S, Strober S., The role of purified CD8+ T cells in graft-versus-leukemia activity and engraftment after allogeneic bone marrow transplantation. Transplantation. 1995; 60:355-361.
57. Drobyski W R, Majewski D., Donor gamma delta T lymphocytes promote allogeneic engraftment across the major histocompatibility barrier in mice. Blood. 1997; 89:1100-1109.
58. Billingham R E., Free skin grafting in mammals. Philadephia: The Wistar Institute Press, 1961.
59. Okabe M, Ikawa M, Kominami K, Nakanishi T, Nishimune Y., Green mice as a source of ubiquitous green cells. FEBS Lett 1997; 407:313-319.
60. DeJamette J B, Sommers C L, Huang K, et al., Specific requirement for CD3epsilon in T cell development. Proc Natl Acad Sci USA. 1998; 95:14909-14914.
61. Dave V P, Cao Z, Browne C, et al., CD3 delta deficiency arrests development of the alpha beta but not the gamma delta T cell lineage. EMBO J. 1997; 16:1360-1370.
62. Wang B Y, Biron C, She J, et al., A block in both early T lymphocyte and natural killer cell development in transgenic mice with high-copy numbers of the human CD3E gene. Proc Natl Acad Sci USA. 1994; 91:9402-9406.
63. Gandy K L, Weissman I L., Characterization of the CD8+ Subpopulations of Whole Bone Marrow That Facilitate Hematopoietic Stem Cells Across Allogeneic Barriers. Blood, Supplement 1. 1996; 88:594a.
64. Corcoran L, Ferrero I, Vremec D, et al., The lymphoid past of mouse plasmacytoid cells and thymic dendritic cells. J Immunol. 2003; 170:4926-4932.
65. Groettrup M, Baron A, Griffiths G, Palacios R, von Boehmer H., T cell receptor (TCR) beta chain homodimers on the surface of immature but not mature alpha, gamma, delta chain deficient T cell lines. EMBO Journal. 1992; 11:2735-2745.
66. Jiang Z, Adams G B, Hanash A M, Scadden D T, Levy R B, The contribution of cytotoxic and noncytotoxic function by donor T-cells that support engraftment after allogeneic bone marrow transplantation. Biol Blood Marrow Transplant. 2002; 8:588-596.

What is claimed is:

1. A method for conditioning a recipient for bone marrow transplantation comprising subjecting the recipient to treatment with a non-lethal dose of body irradiation and an alkylating agent, followed by transplantation with a donor cell preparation containing CD3ε⁺ FC and hematopoietic stem cells from a donor that are matched at the major histocompatibility complex class I K locus with the recipient hematopoietic microenvironment.

2. The method of claim 1 in which the dose is between 1Gy and 7Gy.

3. The method of claim 1, in which the alkylating agent is cyclophosphamide.

4. A cellular composition comprising CD3ε⁺ FC and mammalian hematopoietic stem cells, wherein the hematopoietic stem cells match the recipient hematopoietic microenvironment at the major histocompatibility complex class I K locus.

5. The composition of claim 4, wherein said mammalian hematopoietic stem cells are human.

6. The composition of claim 4, wherein said CD3ε⁺ FC are human.

7. A method of partially or completely reconstituting a mammal's lymphohematopoietic system comprising administering to the mammal the composition of claim 4.

8. The method of claim 7, in which the mammal suffers from autoimmunity.

9. The method of claim 8, in which the autoimmunity is diabetes.

10. The method of claim 8, in which the autoimmunity is multiple sclerosis.

11. The method of claim 8, in which the autoimmunity is sickle cell.

12. The method of claim 8, in which the autoimmunity is anemia.

13. The method of claim 7, in which the mammal suffers from a hematologic malignancy.

14. The method of claim 7, in which the mammal requires a solid organ or cellular transplant.

15. The method of claim 7, in which the mammal suffers from immunodeficiency.

16. The method of claim 7, in which the mammal suffers from cancer.

17. The method of claim 7, in which the mammal suffers from viral infections.

18. The method of claim 7, in which the mammal suffers from metabolic disorders.

19. A method for decreasing the rate of host resistance to the transplantation of hematopoietic stem cells across allogeneic barriers by matching the major histocompatibility complex class I K locus between the donor and the recipient.

20. A cellular composition comprising mammalian hematopoietic stem cells and CD3ε⁺ facilitating cells that are matched at major histocompatibility complex class I K locus.

21. A method for conditioning a donor or recipient for a transplantation by administering 1-30 g/kg per day of Flt3 ligand to an individual.

22. The method of claim 21, wherein said Flt3 ligand is administered for 10 days at a rate of 20 g/kg.

23. A method of partially or completely reconstituting a mammal's lymphohematopoietic system comprising administering to the mammal Flt3 ligand.

24. The method of claim 23, in which the mammal suffers from autoimmunity.

25. The method of claim 24, in which the autoimmunity is diabetes.

26. The method of claim 24, in which the autoimmunity is multiple sclerosis.

27. The method of claim 24, in which the autoimmunity is sickle cell.

28. The method of claim 24, in which the autoimmunity is anemia.

29. The method of claim 23, in which the mammal suffers from a hematologic malignancy.

30. The method of claim 23, in which the mammal requires a solid organ or cellular transplant.

31. The method of claim 23, in which the mammal suffers from immunodeficiency.

32. The method of claim 23, in which the mammal suffers from cancer.
